(19) United States
(12) Patent Application Publication
(10) Pub. No.: US 2010/0041704 A1
(43) Pub. Date: Feb. 18, 2010

(54) DERMAL COMPOSITIONS OF SUBSTITUTED AMIDES AND THE USE THEREOF AS MEDICATION FOR PAIN AND PRURITUS

(76) Inventors: A.K. Gunnar Aberg, Sarasota, FL (US); Keith A. Johnson, Durham, NC (US)

Correspondence Address: Nields, Lemack & Frame, LLC, 176 E. Main Street, Suite #5, Westborough, MA, 01581, US

(21) Appl. No.: 12/448,760

(22) PCT Filed: Jan. 11, 2008
(86) PCT No.: PCT/US08/00425

§ 371 Date: Jul. 6, 2009

Related U.S. Application Data
(60) Provisional application No. 60/880,227, filed on Jan. 12, 2007.

(51) Int. Cl. A61K 31/445; C07D 211/60; A61P 23/00

(52) U.S. Cl. 514/330; 546/225

(57) ABSTRACT

Dermal compositions comprising topical formulations of bupivacaine or ropivacaine, characterized by effective dermal absorption and long duration of dermal anesthetic activity, and intended for use in patients suffering from pruritus and dermal pain, including neuropathic pain, are provided. Compositions containing both bupivacaine and capsaicin are provided. Methods of alleviating pain by the topically administration of these compounds are also provided.

This application claims priority of U.S. Provisional Application No. 60/880,227 filed Jan. 12, 2007, the disclosure of which is incorporated herein by reference.

### TECHNICAL FIELD

This invention relates to dermal and topical pharmaceutical compositions containing racemic or isomeric forms of bupivacaine or ropivacaine (or salts, solvates, polymorphs, etc. thereof), useful, for example, for the treatment of nociceptive pain, neuropathic pain and pruritus, and in particular dermal neuropathic pain.

### BACKGROUND OF THE INVENTION

The present invention refers to pharmaceutically acceptable dermal compositions of the local anesthetic compounds bupivacaine and ropivacaine, the optically active isomers, the salts and the polymorphs thereof. Also included in the present invention are compositions containing combinations of one or more of said local anesthetics and combinations of said local anesthetics with one or more additional therapeutically active entities, such as for example capsaicin, botulinum toxin, one or more steroids and/or one or more analgesic drugs. Included in the invention is also the use of said composition to treat mammals for neuropathic pain, nociceptive pain and pruritus.

Racemic bupivacaine (LAC 43) and racemic ropivacaine (LAC 37) were originally invented and developed at Bofors Nobelkrut/Bofors NobelPharma under the leadership of Dr. Bo af Ekenstam (af Ekenstam B. et al. 1957) and Dr. Fritz Henn (Henn F. 1960). Racemic bupivacaine was originally commercialized under the name Marcaine® by Bofors Nobel-Pharma, levobupivacaine was commercialized under the name Chirocaine® by Chiroscience, and the S-isomer of ropivacaine is commercialized under the names S-Ropivacaine and Ropivacaine by Cipla of Mumbai, India. All of these compounds are local anesthetics, commonly used to inhibit nociceptive pain, and usually administered by injection to obtain infiltration anesthesia or nerve blocks. Topical gel and topical cream formulations of these drugs may exist in various countries for use on mucous membranes, such as for example in the urethra, the mouth and the eye. No significant penetration barriers have to be crossed to obtain topical anesthesia of mucous membranes.

The skin is made up of three layers:

1. Epidermis is the outer layer of the skin, which is made up of five layers. From bottom to top they are: stratum basale, stratum spinosum, stratum granulosum, stratum licidum and stratum corneum. The cells of the stratum corneum constitute the main dermal penetration barrier.

2. Dermis consists of two layers, the upper papillary layer and the lower reticular layer. Blood vessels, nerves and sensory nerve cells are located in dermis.

3. Subcutaneous tissues. Fat, connective tissue, blood vessels and nerves are located in the subcutaneous tissues.

Since bupivacaine as well as ropivacaine cannot easily cross the stratum corneum layer, pharmaceutically acceptable dermal compositions have now been developed that facilitate the dermal absorption of said drugs through intact skin. Both bupivacaine and ropivacaine can easily penetrate through skin, where said penetration barrier has been damaged—as for example after skin grafts have been removed—and simple cream and gel formulations, as for example alginates, may be used in such cases. Thus, uncomplicated topical formulations of bupivacaine have been tried with some success in patients to combat the pain at split skin donor sites, where the normal dermal penetration barrier has been removed or has been seriously damaged (Alvi R et al. 1998), such as for example topical formulations for skin ulcers or burns (Innocoll, 2007). The present invention, however, deals with the absorption of bupivacaine and ropivacaine through intact skin with a functional penetration barrier.

Many standardized commercially available dermal drug delivery systems exist and tests have been described with various devices and delivery systems. In recent studies, bupivacaine was incorporated into gel tablets that were attached to rabbit skin and covered with adhesive film to obtain a patch-like situation (Blanco M D et al 2003). Other devices have also been tried to obtain dermal delivery of bupivacaine, such as microspheres (LeCorre P et al. 1997). All publications and patents mentioned here and elsewhere in this document are hereby included in their entirety by reference.

Dermal applications of the local anesthetic compound lidocaine—in the form of patches—are being used in patients suffering from dermal neuropathic pain. Although useful for certain patients, patches have obvious disadvantages as they fit poorly on a foot or a hand and do not cover the entire dermatome in patients who are suffering from thoracic or abdominal neuropathies, such as for example post-herpetic neuralgia. The use of patches by patients suffering from facial neuropathies, such as for example trigeminal neuropathies, such as for example tic douloureux, have obvious disadvantages. Despite the need for other compositions than patches, no compositions, such as for example gels, ointments, creams or sprays, have successfully been developed for the use of bupivacaine or ropivacaine on intact skin, which we believe is due to the significant difficulties involved in the development of such formulations for these drugs. However, patches may be useful in certain situations, such as for example for systemic or local delivery of medications and such patches, containing bupivacaine and/or ropivacaine are part of the present invention.

The mechanism of action of local anesthetic drugs for the treatment of nociceptive pain, such as for example pain caused by surgery, is a temporary inhibition of nerve conduction in afferent sensory nerves, which is caused by inhibition of ion channels—particularly sodium channels—in said neuronal tissues. It has now been found that the compounds of the present invention have additional pharmacological properties that are setting them apart from other local anesthetics and making them uniquely useful for patients suffering from neuropathic pain. Work has therefore been devoted to the development of pharmaceutically acceptable dermal compositions that will facilitate the penetration of bupivacaine or ropivacaine across the stratum corneum penetration barrier and into intact skin.

The various conditions of neuropathic pain have presented clinicians with multiple challenges, mainly due to a lack of effective treatments. Treatment decisions are often made on a trial and error basis and agents, such as tricyclic antidepressants, gabapentin (Neurontin®, Pfizer), lidocaine patches (Lidoderm®, Endo) narcotics such as oxycodone (OxyContin®, Purdue Frederick) and other painkillers such as tramadol (Ultram®, Ortho-McNeil) are being tried. The development of a better understanding of the pathophysiology of the different neuropathic pain conditions in recent years has made the rational testing possible of compounds that may be useful for neuropathic pain. Better knowledge of various types of neuropathic pain will allow the scientific community to select target indications of drugs for neuropathic pain, such as for example neuropathic post-herpetic neuralgia, low neuropathic back pain, diabetic neuropathic pain, HIV-related neuropathic pain, shingles and various other hyperalgesic and/or neuropathic conditions. Thus, gabapentin and similar compounds are being tried as oral medication for patients suffering from neuropathic low back pain (NLBP), while lidocaine patches may offer temporary relief of post-herpetic neuralgia and diabetic neuropathic pain. Parenteral administration of capsaicin, although causing intense pain upon administration, may offer temporary pain relief. However, NSAIDs (non-steroidal anti-inflammatory drugs), narcotics, and even antidepressants are presently used by patients and doctors who are trying to find medication to decrease the intensity of neuropathic pain.

### SUMMARY OF THE INVENTION

The problems of the prior art have been solved by the present invention, which provides dermal compositions for external use, comprising as the active ingredient a compound selected from the group consisting of 1 butyl-N-(2,6-dimethylphenyl)piperidine-2-carboxamide and 1-propyl-N-(2,6-dimethylphenyl)piperidine-2-carboxamide, or an optically active isomer thereof, or a salt or a solvate or a polymorph thereof, in the form of a topical formulation selected from the group consisting of gels, creams, ointments, aerosols, foams, lotions, pastes, mousse, emulsions and dermal patches. Also disclosed are methods of treating pain and pruritus by administering to patients in need thereof the dermal compositions.

The present invention thus relates to new compositions of the compounds of the present invention, as dermal anesthetics applied on the skin of both animals and humans. Combinations of the chemical entities of the present invention with other therapeutically active molecules, such as capsaicin and steroids, are also disclosed.

Compositions of racemic or isomeric bupivacaine, intended for dermal administration, will preferably contain said drug in its free base form. However, several salt forms, such as for example the monohydrate hydrochloride have suitable solubility in various excipients and have been found to be useful for dermal administration. Formulations such as those disclosed here or similar to those disclosed here, can also be used for various salt forms of the chemical entities of the present invention and the polymorphs thereof.

In certain embodiments, the topical formulation is a solution comprising from about 1 percent to about 20 percent of the active ingredient, together with from 5 percent to 85 percent of ethanol, from 0 percent to 75 percent of propylene glycol, from 0 percent to 20 percent of hexylene glycol, from 0 percent to 20 percent of diisopropyl adipate, from 0 percent to 0.3 percent of chelating agents, from 0 percent to 0.3 percent of antioxidants, from 0 percent to 0.5 percent of preservatives, from 0 percent to 50 percent of penetration enhancers and from 0 percent to 2 percent of capsaicin.

In certain embodiments, the topical formulation is a solution comprising from 1 percent to 20 percent of racemic bupivacaine, from 55 percent to 75 percent of ethanol, 12 percent hexylene glycol, 10 percent diisopropyl adipate, from 0 percent to 0.3 percent of an antioxidant, from 0 percent to 0.5 percent of a preservative and from 0 percent to 2 percent of capsaicin.

In certain embodiment, the topical formulation is a solution comprising percent of racemic bupivacaine, from 70 percent to 73 percent of ethanol, 12 percent hexylene glycol, 10 percent diisopropyl adipate, from 0 percent to 0.3 percent of an antioxidant, from 0 percent to 0.5 percent of a preservative and from 0 percent to 2 percent of capsaicin.

In certain embodiments, the topical formulation is a gel comprising from 1 percent to 20 percent of the active ingredient, from 5 percent to 90 percent of volatile solvents, from 0 percent to 75 percent of non-volatile solvents, from 0.5 percent to 4 percent of polymers, from 0 percent to 0.3 percent of antioxidants, from 0 percent to 0.5 percent of preservatives, from 0 percent to 20 percent of penetration enhancers, from 0 percent to 2 percent of capsaicin, from 0 to 10 percent of a buffer and water to q.s.

In certain embodiments, the topical formulation is a gel comprising from 1 percent to 20 percent of racemic bupivacaine, from 76 percent to 81 percent of ethanol, 12 percent of hexylene glycol, 2 percent of hydroxyethyl cellulose, from 0 percent to 0.3 percent of an antioxidant, from 0 percent to 0.5 percent of a preservative, from 0 percent to 20 percent of penetration enhancers, from 0 percent to 2 percent of capsaicin, from 0 to 10 percent of a buffer and water to q.s

In certain embodiments, the topical formulation is an emollient cream, comprising from 1 percent to 20 percent of the active ingredient, from 0 percent to 50 percent of capric/caprylic triglycerides, from 0 percent to 50 percent of isopropyl myristate, from 0 percent to 25 percent of propylene glycol, from 1 percent to 5 percent of surfactants, from 0.5 percent to 3 percent of polymers, from 0 percent to 0.3 percent of antioxidants, from 0 percent to 0.5 percent of preservatives, from 0 percent to 30 percent of penetration enhancers, from 0 percent to 2 percent of capsaicin, from 0 to 10 percent of a buffer and water to q.s.

In certain embodiments, the topical formulation is an ointment, comprising from 1 percent to 20 percent of the active ingredient, from 2.5 percent to 10 percent of propylene glycol, from 5 percent to 30 percent of microcrystalline wax, from 0 percent to 20 percent of white wax, from 0.5 percent to 5 percent of surfactants, from 0 percent to 0.1 percent antioxidants, from 0 percent to 20 percent of penetration enhancers, from 0 percent to 2 percent of capsaicin, and mineral oil q.s.

In certain embodiments, the topical formulation is an aerosol spray, comprising from 1 percent to 20 percent of the active ingredient, from 0 percent to 40 percent of propylene glycol, from 0 percent to 5 percent of surfactants, from 0 percent to 3 percent of polymers, from 0 percent to 0.3 percent of antioxidants, from 0 percent to 0.5 percent of preservatives, from 0 percent to 40 percent of propellants, from 0 percent to 50 percent of penetration enhancers, from 0 percent to 2 percent of capsaicin, and water or buffered water q.s.

In certain embodiments, the topical formulation is a thermolabile foam, comprising from 1 percent to 20 percent of the active ingredient, from 0 percent to 70 percent ethanol, from 0 percent to 10 percent cetyl alcohol, from 0 percent to 5 percent citric acid, from 0 percent to 10 percent potassium citrate, from 0 percent to 7 percent polysorbates, from 0 percent to 25 percent propylene glycol, from 0 percent to 7 percent stearyl alcohol, from 0 percent to 2 percent of capsaicin and from 0 percent to 3 percent of a corticosteroid.

In certain embodiments, the topical formulation is a thermolabile foam, comprising from 1 percent to 20 percent of bupivacaine, from 0 percent to 70 percent ethanol, from 0 percent to 10 percent cetyl alcohol, from 0 percent to 5 percent citric acid, from 0 percent to 10 percent potassium citrate, from 0 percent to 7 percent polysorbates, from 0 percent to 25 percent propylene glycol, from 0 percent to 7 percent stearyl alcohol, from 0 percent to 2 percent of capsaicin and from 0 percent to 3 percent of a corticosteroid.

In certain embodiments, the topical formulation is an emulsion, comprising from 1 percent to 20 percent of the active ingredient, 5 to 25% mineral oil, 2-8% cetostearyl alcohol, 1-5% ceteth-20, 0-2% dimethicone, 0 to 1% buffer, 0 to 0.5% preservatives, 0 to 0.3% antioxidants and from 0 percent to 2 percent of capsaicin.

In certain embodiments, a patch can be used to enhance transdermal delivery.

### BRIEF DESCRIPTION OF THE DRAWINGS

FIG. 1 is a plot of solubility;

FIG. 2 is a plot of solubility; and

FIG. 3 is plot of solubility versus melting point.

### DETAILED DESCRIPTION OF THE INVENTION

The present invention includes:

Topical formulations of bupivacaine useful for the treatment of neuropathic pain, nociceptive pain and pruritus.

Topical formulations of ropivacaine useful for the treatment of neuropathic pain, nociceptive pain and pruritus.

The formulations of the present inventions may be prepared as solutions, gels, creams, ointments, pastes, mousses, suppositories, aerosol sprays, foams, emulsions and dermal patch compositions.

The use of said formulations in patients suffering from neuropathic pain, nociceptive pain or pruritus

The term “aerosol” as used herein refers to the common definition of the term, which is a dispersion of solid or liquid particles in a gas phase. The terms “aerosol”, “aerosol spray” and “spray” are used interchangeable within this document.

The terms “allodynia” and “allodynic pain” refer to pain from stimuli, which are not normally painful.

The terms “analgesic” and “analgesia” as used herein refer to relief of all types of pain, including acute and chronic forms of nociceptive pain, neuropathic pain and allodynic pain, by drugs that offer analgesic effects, regardless of their mechanism(s) of action.

Terms such as “active ingredient(s)” as used in this document, refers to R-bupivacaine, RS-bupivacaine, S-bupivacaine, R-ropivacaine, RS-ropivacaine and S-ropivacaine as well as any mixtures of R-bupivacaine+S-bupivacaine or R-ropivacaine+S-ropivacaine. Mixtures, including eutectic mixtures using any combination of said compounds are also included in the present invention.

The term “facial neuropathy” refers to dermal neuropathies of the face, most often involving the trigeminal nerve. Tic douloureux is a form of facial neuropathy. Solutions, gels, ointments, creams and thermolabile foams containing an active ingredient are expected to be useful for the treatment of facial neuropathies.

The terms “neuropathic pain” (“neuropathy”) has been defined as pain initiated, or caused, by a primary lesion or dysfunction of the nervous system. The estimated prevalence of this condition is 1.5% of the total population (Taylor, R. S.: Epidemiology of refractory neuropathic pain. Pain Practice, 2006, 6:22-26). The term “neuropathic pain” refers to various syndromes, diseases, disorders and conditions, such as for example post-herpetic neuralgia, trigeminal neuralgia, focal peripheral nerve injury, diabetic neuropathic pain, HIV-related neuropathic pain, chemotherapy related neuropathic pain, neuropathic leg and back pain, fibromyalgia, allodynic pain, phantom (amputation) limb pain and other postsurgical pain syndromes (The Merck Manual, 18th Edition, 2006; see pages 1779-1781 and specific disorders). All types of neuropathic pain are included in the term “neuropathic pain” (“neuropathy”) as used herein.

The term “patient” refers to any mammal in need of care.

The term “nociceptive pain” refers to a normal and expected pain response to an injury or disease outside the nervous system.

The phrase “pharmaceutically acceptable compositions” as used herein implies for example that compositions having an odor, compositions causing pain or pruritus or compositions being cosmetically unpleasant are not pharmaceutically acceptable.

The phrase “pharmaceutically acceptable salt(s),” as used herein includes but is not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmaceutically acceptable anions, including, but not limited to acetate, ascorbate, benzenesulfonate, benzoate, bisulfate, bitartrate, citrate, acid citrate, ethanesulfonate, fumarate, formate, gentisinate, glucaronate, gluconate, glutamate, hydrobromide, hydrochloride, hydroiodide, isonicotinate, lactate, maleate, mesylate, methanesulfonate, nitrate, oleate, oxalate, pamoate, pantothenate, phosphate, acid phosphate, pyrithione, saccharate, salicylate, stearate, succinate, sulfate, tannate, tartrate, p-toluene-sulfonate salts.

The terms “dermal patch”, “transdermal patch” and “patch” are used interchangeably herein and refer to a dermally applied drug delivery system of the general design described in the elsewhere referred Alza patents and intended for systemic delivery of one or more drugs or for delivery of one or more drugs locally into the skin or the tissues below the skin.

The term “pruritus” is synonymous to itch. Pruritus is vastly different from pain as is further discussed later in this document.

The term “solution” refers to aqueous and non-aqueous solutions if not stated differently.

The term “therapeutically sufficient dose” as used herein refers to the daily dose of the active ingredient applied to the skin.

The term “topical anesthesia” as used in this document refers to local anesthesia of mucosal membranes, such as for examples those of the eye, the ear, the mouth, the nose, the rectal area and the urogenital tract. The term “dermal anesthesia” is in this document defined as local anesthesia of the skin. The terms “infiltration anesthesia” and “nerve blocks” refer to the blockade of nerves by parenteral administration (injections, etc.) of local anesthetics.

All publications and patents being referred to in this document are hereby included in their entirety by reference.

The drugs described herein, BUPIVACAINE (Manufacturer: Cipla, Mumbai, India; CAS 14252-80-3), and ROPIVACAINE (Manufacturer: Cipla, Mumbai, India; CAS84057-95-4), are commercially available and the racemate of bupivacaine and the S-isomer of ropivacaine are used therapeutically as inhibitors of nociceptive pain. The S-isomer of bupivacaine may be a registered drug in certain countries. Racemic bupivacaine and racemic ropivacaine can be made using standard methods well known to those skilled in the art. As well known to those skilled in the art of chiral chemistry, the optically active isomers of racemic compounds, like bupivacaine and ropivacaine, can be obtained by chiral resolution methods, using optically active acids, such as for example optically active tartaric acids or analogs thereof. The polymorphs of bupivacaine and ropivacaine can be obtained as described in publications that are referenced herein (Cilurzo et al., 2005 and Bergström et al., 2006).

When used as dermal anesthetics for nociceptive pain, it is important that the composition of these drugs will offer a short onset time of local anesthesia. When used as medication for chronic pain, such as neuropathic pain, there is an emphasis on long duration of pain relief.

As pointed out above, the mechanisms of action are different when these drugs are used for relief of nociceptive pain and neuropathic pain. The mechanism of action of local anesthetic drugs and compositions thereof for the treatment of nociceptive pain, such as for example pain caused by surgery, is a temporary inhibitory effect of nerve conduction in afferent sensory nerves, which is caused by inhibition of sodium channels in said neuronal tissues and which is of significantly longer duration by bupivacaine or ropivacaine than by lidocaine.

The mechanism of action of local anesthetic drugs and compositions thereof for the treatment of neuropathic pain syndromes, such as for example postherpetic neuropathies, are more complex since the painful signals are generated within the neuronal tissues. Thus, neuronal “pacemakers” generate the painful afferent action potentials that are propagated along the afferent nerves. An inhibition of the neuronal pacemaker activity and/or an inhibition of the action potential propagation along an afferent nerve will offer pain relief by mechanism that may be specific for local anesthetic compounds. It has now been shown that bupivacaine potently inhibits neuropathic pain in an animal model and it is assumed that bupivacaine may effectively prevent, inhibit or decrease the pain from neuropathic pain in humans, suffering from such pain.

Pruritus and pain are vastly separate sensory modalities. The reflex response to pruritus (itch) scratching, while the reflex response to pain is withdrawal. Some drugs, such as for example morphine, will cause pruritus but suppress pain. Pruritus is often caused by histamine, activating H-1 receptors in the skin, while dermal nociceptive pain results from activation of free sensory nerve endings in the skin. Actually, individual C-fibers in the cutaneous branch of peroneal nerve are selectively activated by histamine. The mechanisms by which other agents than histamine (such as for example serotonin, certain prostaglandins, PAF, kallikrein, various cytokines, opioids and acetylcholine) are unknown. It is possible that these compounds are causing pruritus by direct mechanisms or by causing or increasing the release of intracellular histamine, improving the affinity of histamine to the H-1 receptors, etc. (Demling R H, et al. 2002, http://www.woundsresearch.com/article/1030). There are multiple types of itch, such as for example itch in connection with atopic dermatitis, post-burn itch, HIV-induced itch, itch in response to insect bites, etc. All types of itch are included in the present invention. Pruritus is a common problem in dogs and cats; pruritus in both mammalian animal species and in humans are included in the present invention. Pain is also a common problem in animals; pain in both mammalian animal species and in humans are included in the present invention.

The presently revealed compositions of bupivacaine and ropivacaine are well suited for the treatment of both neuropathic and nociceptive pain, particularly dermal, neuropathic types of pain, which constitute significant unmet medical needs, particularly in cases where application of patches and similar devices will cause additional pain or where the use of patches is impractical or cumbersome. The use of the present composition in patients suffering from neuropathic pain, nociceptive pain and pruritus are included in the present invention. In addition to the treatment of nociceptive, neuropathic pain, and pruritus, there are many potential uses for effective dermal compositions of bupivacaine and ropivacaine. Thus, medicinal use of dermally applied bupivacaine and/or ropivacaine includes the treatment of burns, contact dermatitis, psoriasis, atopic dermatitis, insect bites, pruritus, skin rash, wounds and other dermal injuries. The present compositions may also be used in the preparation of patients for surgical procedures or for needle injections, such as subcutaneous injections, venipuncture, and in particular for intramuscular or intra joint injections, injections of insulin, injections of corticosteroids and other steroids. The present compositions may also be used in patients in connection with dialysis and particularly recurrent hemodialysis. Said indications are included in the present invention.

Bupivacaine and ropivacaine—by their well documented long duration of action in animals and in humans (Aberg, G et al. 1977)—will offer prolonged therapeutic benefits, such as prolonged pain relief of all types of dermal pain, such as nociceptive pain, including surgical procedures or needle injections, the treatment of burns, contact dermatitis, atopic dermatitis, insect bites, pruritus, skin rash, wounds and other dermal injuries and neuropathic pain such as for example post-herpetic neuralgia, low neuropathic back pain, diabetic neuropathic pain, HIV-related neuropathic pain, shingles and various other hyperalgesic and/or neuropathic conditions.

There are many potential uses for effective dermal compositions of bupivacaine and ropivacaine. Such uses are included in the present invention and include but are not limited to painful dermal ulcers, dermal pain and irritation in connection with ostomy wound management may be treated by use of the presently revealed compositions of bupivacaine. As known to those skilled in the art, painful dermal ulcers affect a variety of patient populations, ranging from patients with decubitus or pressure ulcers (“bedsores”) to patients with venous stasis ulcers. About 25% of sickle cell anemia patients suffer from leg ulcers, which often are chronic (lasting for months or years) and very painful. The present formulations of bupivacaine are very well suited for the treatment of painful dermal pressure ulcers, since in addition to the local anesthetic and the analgesic effects, bupivacaine also has vasodilating properties. Ropivacaine may not have such vasodilating activities, at least not in isolated spinal pial vessels (Lida, H et al. 1997), whereby bupivacaine may be increasing local blood flow and microvascular effects of this these drugs may therefore inhibit the formation of dermal ulcers and improve the healing of dermal ulcers.

The mechanism of action of drugs and compositions thereof for the treatment of neuropathic pain, is not just to offer temporary inhibition of painful afferent action potential, but also to offer analgesic activity, which in this context means that the beneficial (analgesic) effect of said drugs has been found to be of significantly longer duration than their local anesthetic (nerve blocking) duration. The biological mechanisms for said analgesic activity is not known and the analgesic activity may be achieved by numerous and largely unknown mechanisms, such as for example the inhibition of the release of mediators, such as histamine, leukotrienes and/or cytokines, from pro-inflammatory cells. The compounds bupivacaine and ropivacaine have now been found to have such analgesic activities. Treatment of patients suffering from neuropathic pain with compositions of the present invention will lead to relief of pain that may last for several months and even become permanent.

In combination with certain other compounds that influence the function of sensory nerves, such as for example capsaicin, a useful synergism and a surprising potentiation of the therapeutic benefits has been found with regard to the effects on acute pain and particularly with regard to persisting (chronic) pain. Combinations with analgesic drugs, such as for example acetaminophen, or opioid analgesic drugs, such as for example fentanyl or sufentanil will further improve the analgesic activity of bupivacaine or ropivacaine and the formulations of the present invention will allow the dermal co-administration of such drugs.

The therapeutically sufficient dose, which is herein defined as the daily dose of the active ingredient applied to the skin that is necessary to achieve the desired therapeutic effect, depends on numerous factors, such as the size of the area of skin that will be treated with a formulation of the present invention, the anatomical location of the nerves in relation to the surface of the skin, the local concentration barriers in the skin in the area to be treated, the therapeutic effects sought, etc. The amount of the active ingredient necessary to achieve the desired therapeutic effect, or “the therapeutically effective amount”, is an amount that is sufficient to ameliorate, or in some manner reduce or eliminate, pain. A typical topical dose range is from 0.1 mg/cm² to 10 mg/cm² of the composition. Thus, after application of 1 mg/cm² of a composition containing 10 percent bupivacaine, the drug dose would be 0.1 mg/cm². In case the composition is occluded, as is the case with patch formulations, the doses will be lower than if non-occluded compositions are used. As obvious to those skilled in the art, the therapeutically sufficient doses can be considerably higher or considerably lower and will depend on numerous factors, such as the local penetrability of the skin, the frequency of applications, the therapeutic effect sought, etc. It is possible that the applications need not be repeated daily, but every two or three days. It is also possible that certain patients need more than one application daily.

The concentration of bupivacaine or ropivacaine in the present compositions will vary depending on numerous factors, such as for example the penetrability of the skin, the composition used, the therapeutic indication, and the intended frequency of applications. In general, the concentrations of the active ingredient(s) should be 1 percent to 20 percent, but can be lower than 1 percent for example in compositions for burn damage, and may can be higher than 20 percent for example in patients suffering from neuropathic pain.

The compositions of the present inventions may be prepared as solutions, gels, creams, ointments, pastes, mousses, suppositories, sprays, foams, emulsions and as dermal patches

The active ingredients in accordance with certain embodiments of the present invention are N-substituted 2,6-dimethylphenyl-piperidine-2-carboxamides, and the chemical structures of the active ingredients (as their HCl salts) are shown below as Formula 1 and Formula 2.

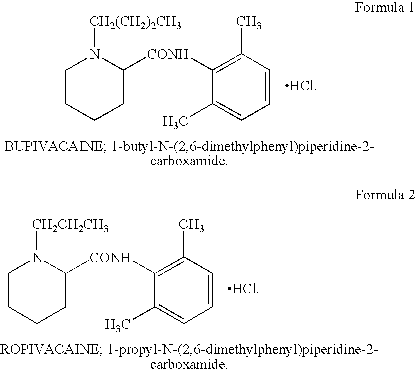

The term “bupivacaine” as used in this document refers to the chemical entity RS—N-(2,6-dimethylphenyl)-1-butylpiperidine-2-carboxamide (also called 1-butyl-N-(2,6-dimethylphenyl)piperidine-2-carboxamide), the single and pure isomers, mixtures of the R- and S-isomers, the polymorphs, the free base, salts, solvates and hydrates of bupivacaine.

The term “ropivacaine” as used in this document refers to the chemical entity RS—N-(2,6-dimethylphenyl)-1-propylpiperidine-2-carboxamide (also called 1-propyl-N-(2,6-dimethylphenyl)piperidine-2-carboxamide), the single and pure isomers, mixtures of the R- and S-isomers, the polymorphs, the free base, salts, solvates and hydrates of ropivacaine. The USAN-name “ropivacaine” refers to the S-isomer of the drug (CAS: 132112-35-7), which is different from the definition used in this document.

The optical purity of the enantiomers of the compounds of the present invention is not particularly limited, but generally should be at least about 80% ee, preferably at least about 90% ee, more preferably at least about 95% ee, most preferably at least about 99% ee.

Polymorphs, solvates and hydrates of amide-type local anesthetics have been described by A. Schmidt, 2005 and are stoichiometric and stable to desolvation. Schmidt also described polymorphs of bupivacaine in said publication. In addition, Cilurzo et al., 2005 described polymorphs of bupivacaine. Other polymorphs may exist.

Polymorphs of ropivacaine have been described (Bergström P-O et al. 2006). Other polymorphs are expected to exist.

Those skilled in the art will realize that the polymorphs may be of limited importance when the compounds of the present invention are dissolved in a composition. As also realized by those skilled in the art, supersaturated solutions may form crystals or fine particles and suspension compositions may also contain crystals and fine particles of the drugs. Said crystals particles may present themselves as polymorphs. All composition containing polymorphs, including for example supersaturated solutions, emulsions and suspensions of the compounds of the present invention are included in the present invention.

Parenteral compositions of the compounds of the present invention have pharmacological properties that render them to be useful as local anesthetics to prevent and to treat pain after injection (U.S. Pat. No. 2,955,111).

The mechanism of action of local anesthetic drugs and compositions thereof for the treatment of nociceptive pain, such as for example pain caused by surgery, is a temporary inhibitory effect of nerve conduction in afferent sensory nerves, which is caused by inhibition of sodium channels in said neuronal tissues. In the case of neuropathic pain the situation is more complex than the case of nociceptive pain, since the painful signals are generated within the neuronal tissues themselves. Thus, neuronal “pacemakers” generate the painful afferent action potentials that are then propagated along the afferent nerves. An inhibition of the afferent action potential propagation with a local anesthetic will offer a temporary relief. An inhibition of the neuronal pacemaker activity will offer pain relief by a different mechanism. It has now been found that bupivacaine after repeated administrations has the ability to produce long-lasting (days not hours) pain relief by unknown mechanisms.

The mechanism of action of drugs and compositions thereof for the treatment of chronic pain, such as for example neuropathic pain, is not just to offer temporary inhibition of afferent nerve conduction, but also to offer analgesic activity, which in this context means that the beneficial (analgesic) effect of said drugs has been found that is of significantly longer duration than the local anesthetic (nerve blocking) duration. The medical reasons for different types of neuropathic pain may vary and may involve an upregulation of neuronal Na+-channels (Hains B. 2003, and The Merck Manual 18^{th} Edition, 2006, page 1779). Although some pharmacological mechanisms have now been defined—such as inhibition of neuronal pacemaker activity—other mechanisms for said analgesic activity are largely unknown. It is believed by us that a factor contributing to analgesic activity of the active ingredients may be due to inhibition of the release of numerous mediators, such as histamine, leukotrienes and/or cytokines, from pro-inflammatory cells. The compounds of Formula 1 (bupivacaine) and of Formula 2 (ropivacaine) have now been found to express such activities.

### Pharmacological Laboratory Studies

1. Neuropathic Pain Animal Studies All local anesthetics offer temporary pain relief and bupivacaine offers long duration of local anesthetic activity. Using the standard nerve ligation model in the rat, we have now surprisingly found that bupivacaine has the ability to offer long-term analgesic relief from neuropathic pain, which is an effect that is not shared by other local anesthetics in the present animal model, where nerve injury was achieved by unilateral ligation of the L5 and L6 spinal roots of the sciatic nerve in rats. The test article was administered systemically three days after the nerve ligation and nociception was measured by observing the presence or absence of tail flick in warm (140° F.) water. Allodynia was measured as paw withdrawal using pinprick methodology with algesimeters, calibrated to a pressure of 5 or 10 grams. A single systemic administration of bupivacaine (0.5%) offered prolonged and complete analgesia (100%) using the tail-flick test method and a prolonged anti-allodynic effect when tested with an algesimeter set at 5 grams.

Test results also indicate that isomeric bupivacaine may be more active than racemic bupivacaine. We have concluded that the generation and/or propagation of the painful neuronal action potentials in neuropathic pain are more sensitive to the ion blocking activity by bupivacaine than by other local anesthetics, but this does not adequately explain the beneficial analgesic effects of repeated (long-term) administration. It is considered to be of importance that administrations of bupivacaine can lead to long-term pain relief, which may be of significant therapeutic importance to patients who suffer from neuropathic pain. Similarly, ropivacaine and lidocaine also had activity in this animal model of neuropathic pain.

The local anesthetic (nerve-blocking) activity of bupivacaine was investigated in separate studies in the rat, where sciatic nerve blocks were performed, followed by challenges with an algesimeter (5 gram) to measure duration of activity. Bupivacaine 0.25% offered sensory nerve block lasting for 70±5 min and 99±7 min after injections of solutions containing bupivacaine HCl 0.25% and 0.50%, respectively. Short-acting local anesthetics, such as mepivacaine and lidocaine offered local anesthesia of approximately 40 min after injections of 0.5% solutions (N=10-12 rats). Blockade of efferent fibers (motor nerve blockade) was observed (for 40 to 75 min) in all studies where local anesthetics were injected close to the sciatic nerve.

The present test results confirmed that bupivacaine offers local anesthesia/nerve blocks of long (hours) duration. Studies of the effects of bupivacaine in the neuropathic pain model, demonstrated that the drug offers additional analgesia of neuropathic pain, even after a single administration and that the analgesic activity of bupivacaine is of a duration, which was a longer duration than that offered by lidocaine and that (4) stereo-selectivity may is present.

### 2. Potentiation Studies

The term “potentiation” as used herein refers to an interaction between two or more drugs or compounds resulting in a pharmacological response greater than the sum of individual responses to each drug or compound (Stedman's Medical Dictionary, 25th Edition. ISBN0-683-07916-6).

A classical pharmacological protocol has been followed as (Group 1) one compound is administered in a dose that is resulting in a sub-maximal pharmacologic effect, (Group 2) a second compound is administered in a dose that is too low to result in any pharmacologic effect, and (Group 3) both compounds are administered together and the resulting pharmacological effect is measured. A reference (control) group (Group 4) of animal administered vehicle only, but no active drug. In the present study, bupivacaine (5% in DMSO/water in a dermal patch, applied for 60 min) was administered a to animals (guinea pigs; N=8) of Group 1. This dose caused dermal anesthesia of a duration of 60±5 min, as measured by pinprick methodology. The Group 2 animals (N=8) were administered capsaicin at a dose that was too low to result in measurable dermal anesthesia and the animals of Group 3 (N=8) were administered a combination of the compounds given to animals of Groups 1 and 2. Group 4 (N=8) of guinea pigs acted as a control group.

The test results demonstrated that the dermal anesthetic activity of a combination of an active dose of bupivacaine+an inactive dose of capsaicin was approximately 50 minutes longer than the dermal anesthetic activity of the active dose of bupivacaine, when given without capsaicin.

A potentiation between the test compounds has therefore now been established.

### 3. Analgesic Activity

The term “analgesic activity”, as used herein, means that the beneficial (analgesic) effect of a local anesthetic is of significantly longer duration than the local anesthetic (nerve blocking) duration of said drug. The biological mechanisms for said analgesic activity are not known and we have postulated that certain anti-inflammatory mechanisms, such as inhibition of the release of mediators, such as histamine, leukotrienes and/or cytokines, from mast cells and other cell types contribute to the analgesia observed.

In the present studies, histamine was the marker compound for algesic mediators that are released into the tissues of patients suffering from neuropathic pain. The inhibition of stimulated histamine release from human leukocytes (buffy coat) by test articles was studied. Leukocytes were obtained from healthy volunteers and histamine release was induced by incubation (20 min/37° C.) with the calcium ionophore A23187 (5 μM) in the presence or absence of a test article. Histamine was analyzed by enzyme-immuno assays, using commercially available kits and a microplate reader (MRX, Dynatech).

Presently available results demonstrate a significant inhibitory activity of bupivacaine on the release of the pain-mediator histamine from human leukocytes. Bupivacaine was more potent than lidocaine.

4. Combinations with Analgesics

Ossipov et al. 1995 have demonstrated beneficial effects of a combination of morphine and bupivacaine in a rat model that was similar to the model described above.

Although the compounds of the present invention and combinations of such a compound with an opioid have proven useful for the treatment of neuropathic pain after injections, the clinical use thereof is limited, since such treatment will cause efferent nerve blockade. Thus, after injections, bupivacaine is causing motor nerve blocks in doses as low as 0.1%. The present invention presents dermal compositions of bupivacaine in combination with at least one other drug with analgesic activity. Such dermal compositions offer relief of long duration from neuropathic pain, without causing efferent nerve function impairment. Non-opioid analgesics, such as for example acetaminophen or COX-1 inhibitors or COX-2 inhibitors and opioid drugs such as for example oxycodon or sufentanil may be used.

### 5. Pruritus Studies

The method used is modified after Makiura et al., 2004. Mice are fed a diet, containing approximately half the content of magnesium and zinc when compared standard laboratory mouse diet. Thus the experimental diet contains about 0.08 percent magnesium and about 60 ppm zinc; all other ingredients are equal to standard laboratory mouse diets. Animals being fed a diet that is low in Mg²⁺ and Zn²⁺ for three to four weeks, develop pruritus, which is characterized by intense scratching of certain areas of the skin. The major affected area of each animal is treated with selected compositions of the present invention, care being taken that the mice do not lick said compositions. While the total mean scratching is increased by about 300 percent in animals fed a diet that is low in Mg²⁺ and Zn²⁺, the scratching of the areas treated with said selected compositions is eliminated or reduced to the low frequency of scratching seen in control animals, fed standard mouse diet. Results obtained to date demonstrate that several compositions of the present invention are safe and effective in animal models of pruritus and do not to cause dermal irritation.

### 6. Dermal Irritation Studies

Dermal irritation studies are performed in male and female New Zealand White rabbits (Harlan). This albino rabbit is recommended as the preferred species because of its size, skin permeability and extensive database. The number of animals being used in these studies is consistently increasing as new compositions are tested and previously tested compositions are being retested as references. Within 24 hours before the initiation of the test, fur is removed from the upper and lower dorsal trunks by clipping. Care is taken to avoid abrading the skin and only animals with healthy intact skin are being used. Histamine is used as the pruritic irritant. The compositions to be tested are applied to the skin of the animals for 60 min (±5 min). The new compositions, with or without bupivacaine 5 to 10 percent, are applied on 6-ply gauze pads (4.5 cm×4.5 cm) and held in place with Tegaderm® dressing for the entire exposure duration. After the exposure period, residual test article is removed with water or other appropriate solvent, without altering the existing response or the integrity of the epidermis. Prior to dosing and repeatedly post dosing, dermal irritation is scored for erythema/eschar and edema.

### Pharmaceutically Acceptable Compositions

The development of compositions for compounds of the present invention has proved to be challenging due to the complicated physicochemical properties of these drugs.

Dermal compositions have now been formulated, such as solutions, creams, gels, ointments and aerosols containing racemic bupivacaine base, racemic bupivacaine salt forms, isomeric bupivacaine base and isomeric bupivacaine salt forms. Dermal compositions have also been formulated for racemic ropivacaine base, racemic ropivacaine salt forms, isomeric ropivacaine base and isomeric ropivacaine salt forms. The new compositions are designed to facilitate the dermal penetration of said molecules, thereby making them useful in the treatment of chronic pain, in particular for the treatment of neuropathic pain.

Said compositions may also contain an additional analgesic agent, such as for example an opioid drug, such as for example morphine or sufentanil or a non-opioid analgesic drug, such as for example acetaminophen or ibuprofen. Said composition may also contain additional ingredients, such as for example botulinum toxin (0.1 to 20 Units; one Unit corresponds to the calculated median intraperitoneal lethal dose in mice) of capsaicin (0.01% to 5%; w/w and preferably 0.075% to 2%; w/w). Said compositions may also contain a second local anesthetic and the mixture between the local anesthetic compounds may be a eutectic mixture. Such eutectic mixtures may have certain advantages when compared with other mixtures of local anesthetics, as know to those skilled in the art, who have knowledge of the advantages of EMLA®, AstraZeneca, which is a eutectic mixture of lidocaine and prilocaine.

The compositions of the invention contain a pre-determined concentration of a compound of the present invention in the form of a free base, in combination with one or more solvents, carriers, penetration enhancers, occlusive agents, and emollients. The combination of excipients provides means of delivering the drug through intact skin or through skin that has been damaged, for example by inflammation (such as for example shingles or atopic dermatitis) or burns or scratching or surgical procedures. Selected compositions will be able to repair dermal barrier damage, protect the skin, and hydrate the skin.

Emphasis was made on dermal safety aspects and multiple new formulations have been subjected to dermal irritation testing, in part using the animal methodology described above.

Compositions of compounds of the present invention in combination with capsaicin have now demonstrated a surprising potentiation of the local anesthetic activity and of the analgesic activity. Not only has the depth of local anesthesia and analgesia been improved, but also the duration of both anesthesia and analgesia were significantly prolonged. Thus, animal experiments have now demonstrated that certain combinations, such as for example a combination of bupivacaine with capsaicin (0.01% to 5%; w/w and preferably 0.075% to 2%; w/w), offer a surprising and significant improvement of the therapeutic activity in animals with induced neuropathies.

Prevention and treatment of (a) nociceptive pain, such as for example pain caused by surgical treatment and (b) neuropathic pain, such as for example postherpetic neuropathic pain and (c) pruritus, using the compositions of this invention may be achieved by applying said compositions in a patch on the skin or by applying carefully designed compositions to the skin, such as for example a spray, a gel, a cream, or an ointment, allowing the therapeutically active ingredient(s) of said composition to pass though the penetrations barriers of the skin and enter into the dermal tissues to offer analgesic activity and to reach the dermal sensory nerves, the sensory nerve endings and various dermal receptors, such as the receptors for heat and cold.

As known to those skilled in the art, the isomers of bupivacaine have certain pharmacological advantages over the racemic compound bupivacaine, such as for example differences in cardiovascular side effects, which may make one isomer more suitable for development than the other, or make one isomer more suitable for development than the racemate. Thus instead of the racemic mixture of bupivacaine, an optically active isomer thereof may be used in the compositions described herein. However, although there are pharmacological reasons to select an isomer of bupivacaine, it has now been found that there are compelling pharmaceutical reasons to select racemic bupivacaine for dermal delivery systems (Table 4).

As is the case with bupivacaine, the optically active S-isomer of ropivacaine has certain pharmacological advantages over the racemate, such as for example less cardiovascular side effects. Thus an isomer of ropivacaine may be more suitable for development than the other, or one isomer may be more suitable for development than the racemate. As is the case with bupivacaine, there are pharmaceutical advantages with racemic ropivacaine over an isomer of this compound.

The therapeutic activity of capsaicin against neuropathic pain is known and a dermal gel containing capsaicin was described in U.S. Pat. No. 5,178,879. Various creams containing capsaicin in concentrations from 0.025 percent to 0.075 percent are commercially available in pharmacies or health stores. Concentrations of capsaicin up to 0.075 percent are only marginally effective (Mason L et al. 2004) and higher concentrations of capsaicin in dermal compositions will activate afferent nerves and cause very significant pain (Iadoarola M J et al. 1998.) The combination of bupivacaine and capsaicin will decrease or completely eliminate the pain (“burning sensation”) caused by capsaicin. Due to the potentiated effects that have now been found, the concentrations of capsaicin can be kept low since the therapeutic activity of capsaicin is potentiated by bupivacaine. On the other hand, it will now be possible to increase the concentrations of capsaicin, since the pain is decreased or eliminated by bupivacaine. Important in this context is that the analgesic activity of bupivacaine is improved by capsaicin and the duration of said analgesia is by bupivacaine is significantly prolonged by capsaicin. The present invention presents that capsaicin can either be administered together with a compound of the present invention in the same composition, or the two therapeutically active entities can be administered separately. Thus, the present invention also provides compositions for treating humans and animals with compositions that contain a combination of a compound of the present composition and another therapeutically active compound.

The ability of bupivacaine to inhibit the pain caused by administration of capsaicin may also be utilized in compositions intended for injections and injectable compositions containing capsaicin in combination with bupivacaine are now expected to offer improved nerve-blocking activity with minor or no pain upon injection and are part of the present invention.

The dermal and topical compositions of the present invention contain bupivacaine or ropivacaine or an optically active isomer thereof in concentrations of about 0.5 percent to 30 percent, preferably 1 percent to 20 percent. Dermal compositions with additional therapeutically active entities may contain capsaicin in concentration of 0.01% to 5%, preferably 0.075% to 2%; w/w. Dermal compositions may also contain sufentanil, fentanyl or any other opioid drug that may be absorbed after dermal application of said compositions. The concentration of fentanyl in the composition may be calculated from the area to be exposed and should allow for an hourly dermal absorption of 5 to 200 microgram, preferably 10 to 100 microgram/hour, resulting in a serum concentration of fentanyl that is therapeutically effective, which is 0.1 to 10 ng/ml. Sufentanil is approximately 5 to 10 times more potent as an analgesic than fentanyl and both drugs have biological half-lives of about seven hours and as realized by those skilled in the art, the doses of sufentanil have to be adjusted accordingly. As realized by those skilled in the art, higher or lower concentrations of both the compounds of the present invention and additional active entities may prove to be useful and, under certain circumstances, even preferred.

Extensive laboratory studies have been performed to identify and develop suitable compositions for the compounds of the present invention and to develop manufacturing methods for said compositions.

Although suitable salt forms of the active ingredients offer therapeutically useful dermal anesthetic effects after dermal applications, dermal compositions containing the free base are often preferred.

Dimethyl sulfoxide (DMSO) is a powerful penetration enhancing compound that can be used in dermal formulation. However, high concentrations of DMSO are causing serious skin damage, including erythema, edema and dermal inflammation, particularly when used together with local anesthetics. As known by those skilled in the art, local anesthetics have limited activity or no activity in inflamed tissues. However, it has now been found that DMSO at lower concentrations (<90%) do not cause increased dermal irritation when used in combination with bupivacaine or ropivacaine and such concentrations of DMSO may therefore be used as an additional excipient in the presently revealed trans-dermal compositions of bupivacaine or ropivacaine. The use of DMSO in non-irritating concentrations together with bupivacaine or ropivacaine is part of the present invention.

### EXAMPLE 1

### Development of Analytical Methods

Bupivacaine and ropivacaine were assayed by HPLC using the method described in Table 1. The reference standard was 0.5 mg/mL prepared from the hydrochloride monohydrate salt for each compound.

TABLE 1
HPLC method for bupivacaine and ropivacaine

| | |
|---|---|
| Analytical Column | XTerra RP₁₈, 5 μm, 150 × 4.6 mm (Waters) |
| Mobile Phase | 65% Acetonitrile/35% phosphate buffer (pH 7.7) |
| Flow Rate | 1.0 mL/min |
| Detection (UV) | 263 nm |
| Injection Volume | 20 μL |
| Column Temperature | Ambient |
| Run Time | 5 minutes |

Four solutions of test articles plus a blank were injected in triplicate. Good linearity for the response was found. The blanks assayed before and after test article solutions showed no significant drug level, which demonstrated there was no carry-over. Based on the reproducibility of results over three orders of magnitude in concentrations of the test articles, good linearity in the anticipated concentration range and no carry-over, the analytical method was considered acceptable to measure solubility of the test articles.

### EXAMPLE 2

### Selection of Pharmaceutically Acceptable Excipients

After determination of solubility parameters of bupivacaine and ropivacaine in a large number of excipients, further studies were performed with selected excipients, which were tested for their capability to dissolve the test compounds. Examples of excipients that were found to be preferred for dermal formulations containing bupivacaine or ropivacaine are shown in Table 2.

Compositions for dermal application may contain one or more excipients in addition to the active ingredient(s). Said compositions may also contain one or more excipients of the same type, meaning that a composition may contain one or more solvents, one or more volatile solvents, one or more emollients, one or more penetration enhancers, one or more humectants, one or more antioxidants, and/or one or more preservatives. Those skilled in the art will realize that other types of excipients may also be found to be useful.

Dermal compositions may also contain one or more surfactants, such as for example ammonium lauryl sulfate, beheneth-10, benzalkonium chloride, ceteth-2, ceteth-10, ceteth-20, ceteth-23, cetyl alcohol, dodecyl betaine, isoceteth-20, hydrogenated lecithin, lanolin alcohol, lecithin, N,N-dimethyldodecylamine N-oxide, oleyl alcohol, peg-40 stearate, peg-100 stearate, peg-6 palm kernel oil esters, peg-6 apricot kernel oil esters, peg-6 corn oil esters, poloxamer 124, poloxamer 182, poloxamer 188, poloxamer 237, poloxamer 407, polyoxyl 60 hydrogenated castor oil, polysorbate 20, polysorbate 40, polysorbate 60, polysorbate 80, polyoxyl 40 hydrogenated castor oil, polyoxyl 75 lanolin, sodium dodecyl sulfate, sodium laureth sulfate sodium dioctyl sulfosuccinate, sodium lauroyl sarcosinate, sorbitan monolaurate, sorbitan monooleate, sorbitan monostearate, sorbitan monopalmitate, sorbitan sesquioleate, stearalkonium chloride and combinations thereof. Extensive compatibility studies demonstrated that preferred surfactants for both bupivacaine and ropivacaine include but are not limited to the polysorbates 20, 40, 60 and 80 and combinations thereof.

Dermal compositions may also contain one or more humectants, which are substances that are used primarily to help retain moisture in cosmetic products and examples are glycerin, sorbitol, polydextrose, propylene glycol, hexylene glycol, sorbitol, and mannitol. Preferred humectants for both bupivacaine and ropivacaine include but are not limited to propylene glycol (0 to 10%) and hexylene glycol (0 to 10%) and combinations thereof.

TABLE 2
Examples of excipients, found to be useful for compositions of
bupivacaine or ropivacaine, such as for example solutions, gels,
ointments, creams and lotions. Those skilled in the art will
appreciate that other excipients may also be found to
be useful. (TGs = triglycerides)

| Excipient | Description | Function |
|---|---|---|
| Capric/caprylic TGs | Semi-polar oil | Oil phase for emulsion, emollient |
| Dibutyl adipate | Semi-polar oil | Solvent, emollient |
| Diisopropyl adipate | Semi-polar oil | Solvent, penetration enhancer |
| DMSO | Solvent | Solvent, penetration enhancer |
| Ethanol | Volatile solvent | Solvent |
| Hexylene glycol | Solvent | Solvent, humectant |
| Isopropyl myristate | Semi-polar oil | Oil phase for emulsion, emollient |
| Isopropanol | Volatile solvent | Solvent |
| Polysorbate 80 | Surfactant | Surfactant |
| Mineral oil | Oil | Oil phase for emulsion, semi- |
| | | occlusive |
| Pentane | Oil | None, used as model propellant |
| Propylene carbonate | Solvent | Solvent |
| Propylene glycol | Solvent | Solvent, penetration enhancer, |
| | | humectant |
| Triacetin | Semi-polar oil | Solvent, Penetration enhancer |
| Water | Water | Solvent, vehicle base, humectant |

### EXAMPLE 3

### Experimental Studies with Bupivacaine

### Materials and Methods

Racemic bupivacaine HCl monohydrate (USP, Spectrum Chemical) and the isomer levobupivacaine HCl monohydrate (Chenghui-Shaungda Chem. Co., Ltd.) were used as received after analysis for identity and purity. All solvents were USP/NF or reagent grade. Acetonitrile was HPLC grade and the phosphate buffer for the mobile phase was prepared with reagent grade salts.

The free bases were prepared by dissolving 5 grams of the salt in 195 g of Purified Water, USP. Approximately 15 mL of 1.0 N NaOH was slowly added until the pH reached 9.5. The precipitate was collected on a filter and washed with water. The solid was re-dispersed in 50 mL of water, mixed for 15 minutes, then filtered and washed with water. The dispersion, filtration, and washing operations were repeated before the material was dried at 40° C. for 72 hours.

Bupivacaine was assayed by HPLC using the method described in Table 1. A reference standard was 0.5 mg/mL bupivacaine free base prepared from the hydrochloride monohydrate salt.

Melting point ranges were determined with a capillary apparatus (Stuart) using a heating rate of 1° C./min. Moisture was measured using Karl Fischer titration (Schott TitroLine KF). Solubility was measured by adding an excess of solid to the solvent and allowing this suspension to equilibrate at 19-21° C. for at least 24 hours. The supernatant was separated by centrifugation, diluted, and assayed by HPLC.

### Results

The free base analysis is summarized in Table 3.

TABLE 3
Properties of bupivacaine free bases prepared in this study.

| Property | Racemate | Isomer |
|---|---|---|
| Yield | 93% | 88% |
| Assay | 101.8% | 100.6% |
| Moisture¹ | <0.36% | <0.28% |
| Melting point range | 105.7-106.8° C. | 136.2-137.0° C. |

¹Using ~100 mg of material, the titration reached equilibrium after the first aliquot of Karl Fischer reagent was added. The pure isomer had an optical purity of 97% ee or better

The average yield for these materials was approximately 90%. The free base was hydrophobic and there was significant adhesion to the beakers and stir bars. The assays were greater than 100% for both materials. The assay values were within usual analytical random errors and may also reflect some purification of the starting materials by recrystallization. After drying for 72 hours at 40° C., the residual moisture was low. The melting point ranges were in agreement with published results. Based on the analytical results, these materials were considered acceptable for solubility testing.

### Solubility Results

Saturation solubility of the active ingredients have been investigated in numerous excipients, as mentioned above. Among the excipients, ethanol and isopropyl alcohol were selected as examples of possible volatile polar solvents. Propylene glycol and hexylene glycol were selected as possible non-volatile polar solvents, which is in addition to these excipients being useful as humectants. Non-polar and semi-polar oils can act as solvents and emollients for the skin and isopropyl myristate and diisopropyl adipate were selected as possible non-volatile semi-polar oils. Other excipients are also useful and can be used instead of or in addition to said selected excipients, (see Table 2), the solubility of which are shown in Table 4, where “Isomer” refers to either the R-isomer or the S-isomer of bupivacaine; as known to those skilled in the art of chiral chemistry, optically active isomers of this type have identical physicochemical properties.

TABLE 4
Solubility of racemic and isomeric bupivacaine free base and salt
in selected topical solvents/penetration enhancers.

| | Solubility of bupivacaine (% w/w) | | | |
|---|---|---|---|---|
| | Racemic free | Isomer free | Racemic HCl | Isomer HCl |
| Solvent | base | base | monohydrate | monohydrate |
| Ethanol | 36.8 | 16.5 | 4.3 | 3.8-4.4¹ |
| Isopropyl | 20.5 | 8.9 | 0.5 | NA |
| alcohol | | | | |
| Hexylene | 17.8 | 8.0 | 2.1 | NA |
| glycol | | | | |
| Propylene | 4.8 | 2.1 | 3.2 | NA |
| glycol | | | | |
| Diisopropyl | 6.2 | 2.7 | NA | NA |
| adipate | | | | |
| Isopropyl | 3.5 | 1.6 | NA | NA |
| myristate | | | | |

¹Determined gravimetrically
NA: Not Available
All single isomer had an optical purity of 97%, ee, or better

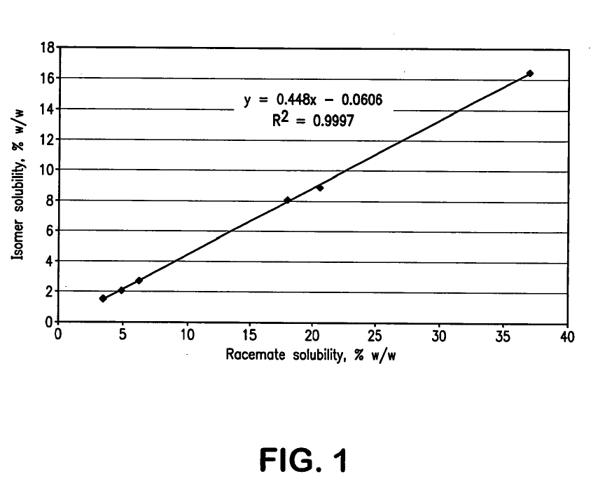

For the free bases, the solubility of the racemate was consistently higher than that for the isomer. FIG. 1 is a plot of the isomer free base's solubility versus that for the racemate for solvents in Table 4. The isomer solubility was consistently lower than the racemate by a factor of approximately 0.45. The solubility difference between the two materials was likely related to their solid-state properties.

In general, the racemic hydrochloride's solubility was lower than either free base. However, the racemic salt's solubility in propylene glycol was higher than the isomer free base and lower than the racemic free base. The water in the hydrochloride's crystal lattice may have contributed to this higher solubility in propylene glycol when compared to the isomer free base. While the salts are very soluble in water, an aqueous vehicle may impede dermal delivery due to free base ionization. The use of ion-paring agents will make the use of salts more practical in compositions and is included in the present invention.

### Conclusions:

Racemic and isomeric free base could be prepared from the hydrochloride salts with acceptable purity and physical properties. The racemic free base had good solubility in all solvents used in this study. The isomer free base solubility was consistently lower than that of the racemate by a factor of approximately 0.45.

Solutions, containing a compound of the present invention are expected to be useful for the treatment of neuropathies, including facial neuropathies.

### EXAMPLE 4

### Experimental Studies with Ropivacaine

### Materials and Methods

(S)-ropivacaine HCl monohydrate (Lot: GLS-FED-060923; chemical and optical purity >98%, GLSynthesis, Worcester, Mass.) was used as received. All solvents were USP/NF or reagent grade. Acetonitrile was HPLC grade and the phosphate buffer for the mobile phase was prepared with reagent grade salts.

Ropivacaine free base was prepared by dissolving 1 gram of the salt in 35 g of Purified Water, USP. Approximately 4 mL of 1.0 N NaOH was slowly added until the pH reached ˜10. The precipitate was collected on a filter and washed with water. The solid was re-dispersed in 50 mL of water, mixed for 15 minutes, then filtered and washed with water. The dispersion, filtration, and washing operations were repeated before the material was dried at 40° C. for 72 hours.

Ropivacaine was assayed by HPLC using the method described in Table 1. The reference standard was 0.5 mg/mL ropivacaine free base prepared from the hydrochloride monohydrate salt.

The melting point range for ropivacaine free base was determined with a capillary apparatus (Stuart) using a heating rate of 1° C./min. Moisture was measured using Karl Fischer titration (Schott TitroLine KF).

Solubility was measured by adding an excess of solid to the solvent and allowing this suspension to equilibrate at 19-21° C. for at least 24 hours. The supernatant was separated by centrifugation, diluted, and assayed by HPLC.

### Results

### Ropivacaine Free Base Preparation.

The free base analysis is summarized in Table 5.

TABLE 5
Properties of ropivacaine free base prepared in this study.

| Property | Value |
|---|---|
| Yield | 91% |
| Assay | 99.4% |
| Moisture¹ | <0.39% |
| Melting point range | 147.1-147.9° C. |

¹Using ~100 mg of material, the titration reached equilibrium after the first aliquot of Karl Fischer reagent was added.

The assay values were within usual analytical random errors. After drying for 72 hours at 40° C., the residual moisture was low. The melting point range was higher than that for the bupivacaine free bases. Based on the analytical results, this material was considered acceptable for solubility testing.

### Solubility Results

Ethanol and isopropyl alcohol were selected as possible volatile polar solvents. Hexylene glycol and propylene glycol were selected as possible non-volatile polar solvents, which are in addition to these excipients having other useful properties, such as for examples as humectants. Non-polar and semi-polar oils can act as solvents and emollients for the skin. Diisopropyl adipate and isopropyl myristate were selected as possible non-volatile semi-polar oils. Solubility results are shown in Table 6.

TABLE 6
Solubility of S-ropivacaine free base in several topical
solvents/penetration enhancers.

| | Solubility, % |
|---|---|
| Solvent | w/w |
| Ethanol | 10.0 |
| Isopropyl alcohol | 5.2 |
| Hexylene glycol | 5.1 |
| Propylene glycol | 1.6 |
| Diisopropyl adipate | 1.7 |
| Isopropyl myristate | 0.9 |

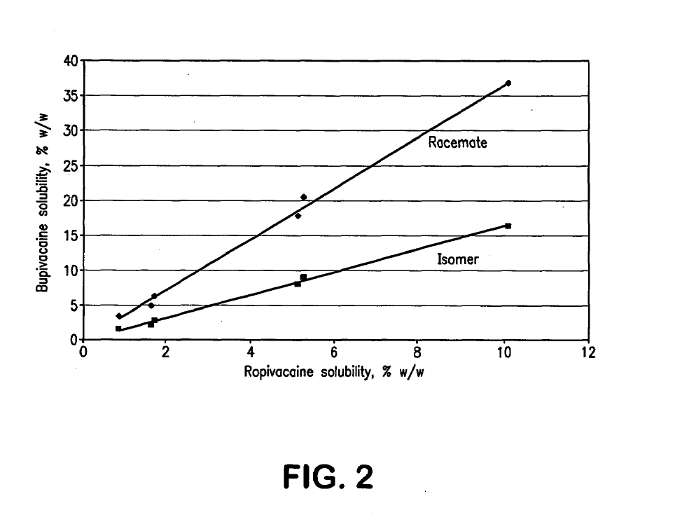

The solubility of the ropivacaine free base was consistently lower than the corresponding solubility for bupivacaine free base. FIG. 2 is a plot of free base solubility of both compounds for all the solvents in Table 6. Even though the range of solvent physical properties was quite wide, the solubility for the three materials—racemic bupivacaine, isomeric bupivacaine and isomeric ropivacaine—had a good correlation. The solubility difference between the materials was likely related to their solid-state properties.

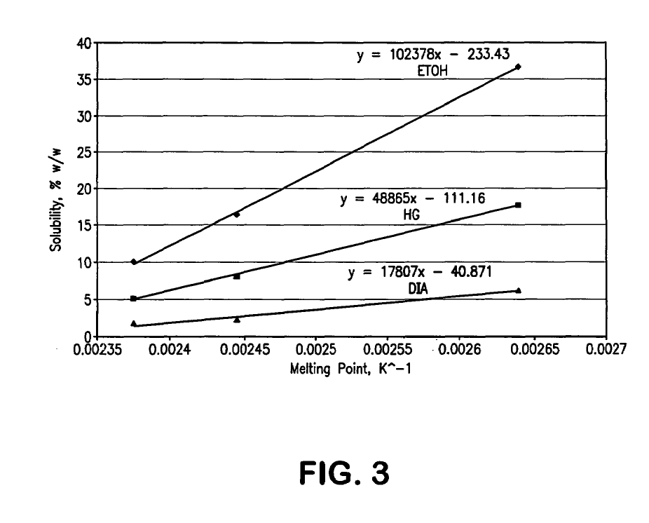

FIG. 3 shows the correlation between solubility and the reciprocal of melting point, in K⁻¹, for the three free bases in three solvents. The solvents selected were the most active solubilizers for each class: polar-volatile (ethanol), polar-non-volatile (hexylene glycol), and semi-polar oil (diisopropyl adipate). The correlation was good and it is concluded that for these compounds, solid-state properties were key factors for solubility and racemic bupivacaine was clearly more soluble than either of the tested isomers.

### Conclusions

Ropivacaine free base was prepared and had lower solubility than either of racemic bupivacaine free bases or isomeric bupivacaine in any given solvent. The solubility of the free bases in a given solvent appeared to be well correlated with the melting points of the compounds.

Although not tested here, those skilled in the art will conclude from the present test results that the solubility of racemic ropivacaine is higher than for isomeric ropivacaine.

### EXAMPLE 5

### Prototype Dermal Solution Compositions and Gel Compositions of Bupivacaine and Ropivacaine

A significant number of prototype dermal compositions of bupivacaine and of ropivacaine have been prepared and tested. Selected examples are shown in Table 7.

TABLE 7
Four selected prototype topical compositions (% as w/w).

| | Composition | | | |
|---|---|---|---|---|
| Component | 61216-1A | 61216-1B | 61216-2 | 61216-3 |
| Bupivacaine free base, | 5% | 5% | — | — |
| racemate | | | | |
| Bupivacaine free base, isomer | — | — | 5% | — |
| Ropivacaine free base, isomer | — | — | — | 2.5% |
| Ethanol | 81% | 73% | 73% | 82.5% |
| Hexylene glycol | 12% | 12% | 12% | 10% |
| Propylene glycol | — | — | — | 5% |
| Diisopropyl adipate | — | 10% | 10% | — |
| Hydroxyethyl cellulose | 2% | — | — | — |

The composition 61216-1A is a clear gel while the remaining three compositions are solutions. It was found that gel compositions corresponding to 61216-2 and 61216-3 can be prepared with the addition of hydroxyethyl cellulose or other compatible gelling compounds.

### EXAMPLE 6

### Tests of Ternary Solvent Mixtures

Laboratory studies have now demonstrated the compatibility of combinations compounds of the present invention in various solvents with water as an anti-solvent. Thus, the solubility of ropivacaine free base in a mixture of 1 ml of polyethylene glycol and 1 ml of ethanol is dramatically decreased by the presence of only 0.6 ml of the anti-solvent (water).

Suitable dermal compositions of the compounds of the present invention include (1) solutions, (2) gels, (3) creams or lotions, (4) ointments, (5) suppositories.

### EXAMPLE 7

### Aqueous and Non-Aqueous Solutions

Solutions of active ingredients for dermal and topical applications. The solutions of the compounds may be useful for various indications, such as for example by patients suffering from neuropathic pain, painful ocular conditions, etc. The concentration of the active ingredients in said solutions may be limited by their solubility as discussed above.

TABLE 8
Solutions of the active ingredients, and the optically active isomers
thereof for dermal and topical applications:

| | % w/w of |
|---|---|
| Component | Composition |
| Bupivacaine or Ropivacaine | 1 to 30 |
| Ethanol | 5 to 85 |
| Propylene glycol | 0 to 75 |
| Antioxidants^{1 0 to 0.3} | |
| Preservatives² | 0 to 0.5 |
| Penetration enhancers³ | 0 to 50 |
| Chelating agent⁴ | 0 to 0.3 |
| Water/buffer⁵ | q.s. |

¹Examples: Butylated hydroxyanisole (BHA), butylated hydroxytoluene (BHT)
²Examples: ethyl paraben, propyl paraben, imidurea
³Examples: DMSO, diethylene glycol monoethyl ether, hexylene glycol, diisopropyl adipate, n-decyl methyl sulfoxide, dimethylacetamide, laurocapram (Azone ®), dimethylformamide, sucrose monooleate, N-methyl-2-pyrrolidine (Pharmasolve ®), oleic acid.
⁴Examples: EDTA (ethylenediaminetetraacetate), Ca-EDTA
⁵Most often 0 to 10 percent w/w of the buffer and then water to q.s.

The dermal compositions will preferably contain from 0.1 percent to 30 percent of an active ingredient in its free base form although a pharmaceutically acceptable salt form may also be used.

Capsaicin (0.01% to 5%; w/w and preferably 0.075% to 2%; w/w) can be included in all compositions of the present invention to obtain prolonged relief from pain and in particular from neuropathic pain, since compound of the present invention will inhibit or decrease the initial pain caused by capsaicin, since the onset time of local anesthesia will become shortened and since analgesic potentiation of depth and/or duration has now been found to take place. Other capsaicinoids, such as for example dihydrocapsaicin, nordihydrocapsaicin, homocapsaicin homodihydrocapsaicin can also be used in analogous amounts, as can the partial capsaicin receptor antagonist capsazepine and other compounds that directly increase the inflow of calcium through the neuron membranes or indirectly influence ionic flow through neuron membranes through activation of the vanilloid subtype-1 receptor (such as for example resiniferatoxin).

### EXAMPLE 8

### Binary Solvent Mixtures with Water

Once a vehicle is applied to the skin, a significant portion of any water in the composition is likely to evaporate. For non-aqueous compositions or compositions with high solvent levels, excipients may also extract moisture from the skin and environment. Therefore, the impact of water on solubility in some water-soluble excipients was investigated.

### EXAMPLE 9

### Methods of Manufacture

### Solutions for Dermal and Topical Application

Dissolve the active ingredient in the excipient solvent where it has the highest solubility (usually ethanol). Add additional solvents with preservatives, penetration enhancers, and antioxidants; then mix until well combined. Add water with buffers as needed. Mix until well combined then pack into appropriate container/closure system.

The free base or a pharmaceutically acceptable salt of the racemic form of bupivacaine or ropivacaine or an optically active isomer thereof can be used; a polymorph, solvate or hydrate thereof may also be used. The solutions will preferably contain from 0.1 percent to 30 percent of the compound of the present invention.

### EXAMPLE 10

### Aqueous or Non-Aqueous Gels

A dermal gel composition of bupivacaine or ropivacaine can contain polyethylene glycol (PG), water and/or buffer, ethanol, a polymer, and optionally another penetration enhancer. Table 7 gives an example of a gel composition (OWL-61216-1). Since the amount of water in a gel composition may be small, polymers that can gel non-aqueous system such as hydroxypropylcellulose (HPC) or hydroxyethylcellulose (HEC) can be used. In experiments with HEC, this excipient was found not only to ably gel the systems, but HEC was also found to be able to stabilize supersaturated solutions of bupivacaine on the skin's surface. As persons skilled in the art will realize, other topically acceptable gelling agents and may also prove to be useful in gels containing bupivacaine and/or ropivacaine. The concentrations of the active ingredient—particularly the concentration of ropivacaine—in the gels, and in any other formulation, may be limited by their solubility as discussed above.

Gels, containing a compound of the present invention are expected to be useful for the treatment of pruritic conditions and pain, particularly neuropathic pain, including facial neuropathies.

TABLE 9
Example of gels containing bupivacaine and/or ropivacaine base for
dermal applications:

| | % w/w of |
|---|---|
| Component | Composition |
| Compound(s) of the | 1 to 30 |
| invention | |
| Volatile solvent¹ | 5 to 90 |
| Solvents² | 0 to 75 |
| Polymers³ | 0.5 to 4 |
| Antioxidants⁴ | 0 to 0.3 |
| Preservatives⁵ | 0 to 0.5 |
| Penetration enhancers⁶ | 0 to 20 |
| Water/buffer⁷ | q.s. |

¹Examples: Ethanol, isopropanol
²Examples: DMSO, propylene glycol, hexylene glycol, propylene carbonate
³Examples: Hydroxypropylcellulose, hydroxyethylcellulose, carbomer
⁴Examples: Butylated hydroxyanisole (BHA), butylated hydroxytoluene (BHT)
⁵Examples: Methyl paraben, propyl paraben, imidurea
⁶Examples: DMSO, diisopropyl adipate. diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), oleic acid
⁷Most often 0 to 10 percent w/w of the buffer and then water to q.s.

Capsaicin (0.01% to 2%; w/w) can be included into said gels to obtain a pain-free drug, offering a prolonged relief from pain, including neuropathic pain.

### Methods of Manufacture: Gels

Dissolve the active ingredient in the solvent where it has the highest solubility (usually ethanol). Add additional solvents with preservatives, penetration enhancers, and antioxidants then mix until well combined. Disperse polymer under appropriate conditions recommended by the manufacturer. Add water with buffers (if required) and neutralizing bases as needed. Mix until well combined and thickened then pack into appropriate container/closure system. The racemic form of bupivacaine or an optically active isomer thereof can be used for aerosols. The free base or a suitable salt form can be used.

The free base or a pharmaceutically acceptable salt of the racemic form of bupivacaine or ropivacaine or an optically active isomer thereof can be used; a polymorph, solvate or hydrate thereof may also be used. The gels will preferably contain from 0.1 percent to 30 percent of the compound of the present invention.

### EXAMPLE 11

### Emollient Creams

The solubility of bupivacaine in emollient oils was exploited to develop elegant cream compositions. When the oil-phase was approximately 50%, a bupivacaine cream could be made with isopropyl myristate or capric/caprylic triglycerides. Additional excipients can also be added as shown in Table 10. The concentrations of the active ingredients in the creams may be limited by their solubility as discussed above.

TABLE 10
Examples of creams containing bupivacaine and/or ropivacaine
base for dermal applications:

| | % w/w of |
|---|---|
| Component | Composition |
| Active ingredient(s) | 1 to 30 |
| Capric/Caprylic Triglycerides | 0 to 50 |
| Isopropyl myristate | 0 to 50 |
| Propylene glycol | 0 to 25 |
| Surfactants | 1 to 5 |
| Polymers¹ | 0.5 to 3 |
| Antioxidants² | 0 to 0.3 |
| Preservatives³ | 0 to 0.5 |
| Penetration enhancers⁴ | 0 to 30 |
| Water/buffer⁵ | q.s. |

¹Examples: Hydroxypropylcellulose, hydroxyethylcellulose, carbomer, xanthan gum
²Examples: Butylated hydroxyanisole (BHA), butylated hydroxytoluene (BHT)
³Examples: Methyl paraben, propyl paraben, imidurea
⁴Examples: DMSO, diethylene glycol monoethyl ether, n-decyl methyl sulfoxide, dimethylacetamide, diisopropyl adipate, laurocapram (Azone ®), dimethylformamide, sucrose monooleate, N-methyl-2-pyrrolidine (Pharmasolve ®), oleic acid
⁵Most often 0 to 10 percent w/w of the buffer and then water to q.s.

Capsaicin (0.01% to 5%; w/w and preferably 0.075% to 2%; w/w) can be included into said creams to obtain a pain-free drug, offering a prolonged relief from pain, including neuropathic pain.

Creams containing a compound of the present invention are expected to be useful for the treatment of neuropathies, including facial neuropathies.

### Methods of Manufacture: Creams

Combine oils, semi-polar oils, antioxidants, penetration enhancers, and surfactants and heat to 60-75° C. (Phase A). Combine polar solvents, preservatives, water, buffer salts, and polymer and heat to 60-75° C. (Phase B). Dissolve the active ingredient in Phase A then immediately add Phase A to Phase B with rapid stirring. High-shear mixing for a specified time may also be required. Cool the mixture while stirring to 30-50° C. If the polymer requires a basic substance to thicken (e.g., sodium hydroxide or triethanolamine), add the base. Once product is sufficiently thickened and cool (≦35° C.), stop stirring and begin packing the product into the appropriate container closure system. The racemic form of bupivacaine or an optically active isomer thereof can be used for aerosols. The free base or a pharmaceutically acceptable salt of the racemic form of bupivacaine or ropivacaine or an optically active isomer thereof can be used; a polymorph, solvate or hydrate thereof may also be used. The creams will preferably contain from 0.1 percent to 30 percent of the compound of the present invention.

### EXAMPLE 12

### Ointments for Dermal, Urethral, Rectal and Vaginal Application

The solubility of bupivacaine in “oily” excipients like isopropyl myristate (IPM) and mineral oil can be seen as an advantage since such excipients tend to be non-irritating and may be suited for ointments compositions, which can be used for various indications, such as for example nociceptive pain, pruritic conditions and neuropathic pain. The concentrations of the active ingredients may be limited by their solubility as discussed above.

TABLE 11
Ointments containing bupivacaine and/or ropivacaine.

| Component | % w/w of Composition |
|---|---|
| Active ingredient(s) | 1 to 30 |
| Propylene glycol | 2.5 to 10.0 |
| Microcrystalline wax | 5.0 to 30.0 |
| White wax (bees wax) | 0.0 to 20.0 |
| Surfactants | 0.5 to 5.0 |
| Antioxidants¹ | 0.0 to 0.1 |
| Penetration enhancer² | 0.0 to 20.0 |
| Mineral oil | q.s. |

¹Examples: Butylated hydroxyanisole (BHA), butylated hydroxytoluene (BHT)
²Examples: DMSO, diethylene glycol monoethyl ether, n-decyl methyl sulfoxide, dimethylacetamide, diisopropyl adipate, laurocapram (Azone ®), dimethylformamide, sucrose monooleate, N-methyl-2-pyrrolidine (Pharmasolve ®), oleic acid

Capsaicin (0.01% to 5%; w/w and preferably 0.075% to 2%; w/w) can be included into said ointments to obtain a pain-free drug, offering a prolonged relief from pain, such as neuropathic pain.

Ointments, containing a compound of the present invention, are expected to be useful for the treatment of neuropathies, including facial neuropathies.

### Methods of Manufacture: Ointments

Combine waxes, oils, and semi-polar oils, and antioxidants and heat to 60-75° C. (Phase A). Combine the active ingredient with solvent (e.g., propylene glycol) and penetration enhancer (if used) and heat to 60-75° C. (Phase B). Add Phase B to Phase A and mix under high shear while cooling. Once ointment thickens, stop high shear and mix with low shear until product is sufficiently cool (≦35° C.), stop stirring and begin packing the product into the appropriate container closure system. The free base or a pharmaceutically acceptable salt of the racemic form of bupivacaine or ropivacaine or an optically active isomer thereof can be used; a polymorph, solvate or hydrate thereof may also be used. The ointments will preferably contain from 0.1 percent to 30 percent of the active ingredient.

### EXAMPLE 13

### Suppositories for Rectal or Vaginal Applications

Suppositories are useful for the treatment of vaginal and rectal painful conditions.

TABLE 12
Suppository compositions containing bupivacaine or ropivacaine
base for vaginal applications.

| Component | % w/w of Composition |
|---|---|
| Active ingredient(s) | 1 to 20 |
| Antioxidants¹ | 0.0 to 0.3 |
| Preservatives² | 0.0 to 0.5 |
| Penetration enhancers³ | 0.0 to 10.0 |
| Suppository maxtrix⁴ | q.s. |

¹Examples: Butylated hydroxyanisole (BHA), butylated hydroxytoluene (BHT)
²Examples: Methyl paraben, propyl paraben, imidurea
³Examples: DMSO, diethylene glycol monoethyl ether, n-decyl methyl sulfoxide, dimethylacetamide, diisopropyl adipate, laurocapram (Azone ®), dimethylformamide, sucrose monooleate, N-methyl-2-pyrrolidine (Pharmasolve ®), oleic acid
⁴Polyethylene glycol (molecular weights 200 to 20,000), hydrogenated vegetable oil (e.g., Wecobee ® S, Wecobee ® FS

Capsaicin (0.01% to 5%; w/w and preferably 0.075% to 2%; w/w) and/or a corticosteroid (0.1% to 3%) can be included into said suppositories to obtain a pain-free drug, offering a prolonged relief from pain, including neuropathic pain.

Rectal and vaginal suppositories, containing an active ingredient, are expected to be useful for the treatment of hemorrhoidal pain, rectal and vaginal pruritic conditions and urogenital neuropathies.

### Methods of Manufacture: Suppositories

Melt suppository matrix material (i.e., polyethylene glycol or partially hydrogenated vegetable oil) then dissolve the active ingredient in the melt. Fill melt into mold, cool, and un-mold. Pack suppositories in appropriate container/closure system. The free base or a pharmaceutically acceptable salt of the racemic form of bupivacaine or ropivacaine or an optically active isomer thereof can be used; a polymorph, solvate or hydrate thereof may also be used. The suppositories will preferably contain from 0.1 percent to 30 percent of the active ingredient.

### EXAMPLE 14

### Aerosol Sprays

### Pump or Propellant Driven

In addition to the solubility experiments shown above, the solubilities of bupivacaine and ropivacaine were also investigated using pentane since this is a low molecular weight hydrocarbon, commonly used to model a GRAS (generally recognized as safe) aerosol propellant such as butane. The result is shown below. The surprisingly high solubility of both bupivacaine and ropivacaine in pentane indicates that hydrocarbon aerosol propellants such as butane will be useful for drug delivery to the skin by aerosol.

TABLE 13
Saturation solubility of bupivacaine (base) and ropivacaine (base)
in pentane.

| | BUPIVACAINE | ROPIVACAINE |
|---|---|---|
| Solvent | Percent | Percent |
| Pentane | >0.1 | >0.1 |

Since bupivacaine and ropivacaine are soluble in both volatile solvents and low molecular weight hydrocarbons, aerosol compositions are possible. The aerosols will contain a volatile solvent or propellant along with a non-volatile solvent plus optionally one or more penetration enhancers. When ethanol is the volatile solvent, a pump spray will be required to create an aerosol. When butane is used, the container/closure system can be pressurized and only a metering value is required to create a useful aerosol administration system.

TABLE 14
Aerosol compositions of bupivacaine and/or ropivacaine
(propellant driven or pump-driven)

| Component | % w/w of Composition |
|---|---|
| Active ingredient(s) | 1 to 30 |
| Propylene glycol | 0 to 40 |
| Surfactants | 0 to 5 |
| Polymers¹ | 0 to 3 |
| Antioxidants² | 0 to 0.3 |
| Preservatives³ | 0 to 0.5 |
| Propellants⁴ | 0 to 40 |
| Penetration enhancers⁵ | 0 to 50 |
| Water/buffer⁶ | q.s. |

¹Examples: Hydroxypropylcellulose, hydroxyethylcellulose, carbomer
²Examples: Butylated hydroxyanisole (BHA), butylated hydroxytoluene (BHT)
³Examples: Methyl paraben, propyl paraben, imidurea
⁴Examples: Butane, isobutane, propane, hydrofluoralkane (134a, 227)
⁵Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid.
⁶Most often 0 to 10 percent w/w of the buffer and then water to q.s.

Capsaicin (0.01% to 5%; w/w and preferably 0.075% to 2%; w/w) can be included into said aerosols to obtain a pain-free composition, offering a prolonged relief from pain, including neuropathic pain and from pruritus.

Aerosols, containing a compound of the present invention, are expected to be useful for the treatment of neuropathies, including facial neuropathies, and for the treatment of pruritus.

### Methods of Manufacture: Aerosols

If the product is a pump spray, prepare solution as described in the solutions section and fill into the appropriate container/closure system with spray pump.

If the product is a propellant-driven spray, dissolve one or more of the active ingredients in liquefied propellant(s) and add appropriate solvents, penetration enhancers, surfactants and polymers. Mix until homogeneous then fill under pressure into the appropriate container/closure system. Alternatively, one or more of the active ingredients plus solvents, surfactants/polymers, and penetration enhancers can be combined at room temperature/pressure then filled into an aerosol canister. After crimping the metering valve in place, the propellant can be pressure-filled into the container. The free base or a pharmaceutically acceptable salt of the racemic form of bupivacaine or ropivacaine or an optically active isomer thereof can be used; a polymorph, solvate or hydrate thereof may also be used. The aerosol solutions will preferably contain from 0.1 percent to 30 percent of the active ingredient(s).

### EXAMPLE 15

### Foams

Foams and in particular thermolabile foams may be used as dermal compositions and may be particularly useful to treat pain and/or pruritic conditions and in particular pruritic conditions affecting the scalp, since we have demonstrated antipruritic activity of bupivacaine and ropivacaine in an animal model. The foam contains one or more of the active ingredients in concentrations ranging from 1% to 30% (of the composition), in combination with ethanol (in concentrations from 0% to about 70% in water), cetyl alcohol (from 0% to 10%), citric acid (from 0% to 5%), potassium citrate (from 0% to 10%), a polysorbate (usually polysorbate 60; from 0% to 7%), propylene glycol (0% to 25%) and stearyl alcohol (0% to 7%). The composition is contained in an aluminum can, pressurized with a commercially available and regulatory approvable propellant. Scalp itch is a highly prevalent condition and there are many possible causes for this condition, ranging from seborrheic dermatitis and psoriasis to stress-induced scalp itch. Thermolabile foam containing an active ingredient may contain additional ingredients that are therapeutically active, such as for example a corticosteroid, such as hydrocortisone in concentrations ranging from 0.1 percent to 3 percent, preferably about 1 percent of the composition.

Capsaicin (0.01% to 5%; w/w and preferably 0.075% to 2%; w/w) can be included into said foams to obtain a pain-free composition, offering a prolonged relief from pain, including neuropathic pain and from pruritus.

When used for the treatment of pruritus, foam and any other composition of the present invention may have additional anti-pruritic agents included in said compositions. Examples of antipruritic agents that can be included in compositions of the invention are steroids (as for example hydrocortisone; 0.1 percent to 5 percent), immunosuppressants (as for example tacrolimus; 0.03 percent to 0.1 percent), etretinate/acetritin (0.1 percent to 10 percent) and zinc pyrithione (0.1 percent to 3 percent).

Foams, including thermolabile foams, containing an active ingredient, are expected to be useful for the treatment of neuropathies, including facial neuropathies and pruritus.

### Methods of Manufacture: Thermolabile Foams

Thermolabile foams can be prepared by incorporating self-warming excipients that are exothermic when exposed to water and/or air. Examples of such excipients are, but are not limited to, polyethylene glycol 200, polyethylene glycol 300, and bentonite. The foam formulation in the container/closure system is essentially nonaqueous. When the foam is applied to the skin, the self-warming excipient(s) will release heat when exposed to the skin surface. Additional water may be applied to the application site to increase the heat released. The water could be incorporated into the foam at the time of application by using a two compartment dispenser: one compartment contains the nonaqueous formulation with self-warming excipients and another compartment contains water or an aqueous formulation. The contents of each compartment are released and mixed in the dispenser when activated by the user. The product is a thermolabile foam that can applied to the skin.

The free base or a pharmaceutically acceptable salt of the racemic form of bupivacaine or ropivacaine or an optically active isomer thereof can be used; a polymorph, solvate or hydrate thereof may also be used. The foams will preferably contain from 1 percent to 30 percent of the active ingredient(s).

### EXAMPLE 16

### Emulsions

The present invention also includes emulsion, containing at least one of the active ingredients, but optionally more than one compound and optionally a eutectic mixture between active ingredients of between an active ingredient and another local anesthetic that is not any of the compounds of the present invention. Examples of other local anesthetics, that can be used in any composition (not only emulsions) described here, together with an active ingredient are lidocaine, prilocalne, etidocaine, mepivacaine and tetracaine.

An emulsion is a mixture of two or more immiscible phases, such as a lipid-soluble substance dispersed in a water phase. Emulsions are unstable and tend to revert to the stable state of the lipid separated from the water phase. Surface active substances (surfactants) can increase the kinetic stability of emulsions and the inclusion of a surfactant may add years to the shelflife of an emulsion.

Emulsions can exist as watery mixtures, but a preferred emulsion composition is a gelled emulsion with the lipid-soluble local anesthetic base and the aqueous phase being mixed with gelling agent(s) and/or emulsifying agents.

A gelled emulsion may stay as a gel or may soften or melt by body heat or external heating, depending on the gelling agent(s) and/or the thickening agent(s) used.

An example of an emulsion may contain 5 to 25 percent of an oil phase containing one or more local anesthetic and 80 to 95 percent of the aqueous phase, a gelling agent such as for example carrageenan at about 5 percent and one or more surfactant(s), preservatives, etc.

An emulsion formulation for may comprise from 1 percent to about 20 percent of said N-substituted 2,6-dimethylphenyl-piperidine-carboxamide, 5 to 25% mineral oil, 2-8% cetostearyl alcohol, 1-5% ceteth-20, 0-2% dimethicone, 0 to 1% buffer, 0 to 0.5% preservatives, 0 to 0.3% antioxidants and from 0 percent to 2 percent of capsaicin. Cetostearyl alcohol is a moisturizer, including cetyl and stearyl alcohol. Ceteth-20 (a surfactant) is the polyethylene glycol ether of cetyl alcohol, where n has an average value of 20. Dimethicone is an organic silicone, used as skin protectant, emollient, moisturizer and antifoam agent. The buffer used in emulsions and in other compositions of the present invention may be a phosphate buffer, pH 7.1-7.6, preferably pH 7.3-7.4.

The term emulsion, as used herein, includes nanoemulsions (emulsions in which the sizes of the particles in the dispersed phase are defined as less than 1000 nanometers).

Emulsions are often presented in the forms of creams.

### Methods of Manufacture: Emulsions

The emulsion can be prepared by combining and heating, if necessary, the nonaqueous ingredients to a suitable temperature (25-80° C.) so that all the components are melted. In a separate container, the water-soluble excipients are combined and brought to approximately the same temperature as the nonaqueous components. Once the temperatures of both phases are correct, they are combined with sufficient mixing to products fine droplets of the dispersed phase in the continuous phase. The emulsion is cooled if necessary. If a gelling agent is required, it can be added during the process.

The free base could be dissolved in an oil and made into an emulsion. A salt could also be used by dissolving it in the aqueous phase.

Suitable penetration enhancers for use with the active ingredients include, but are not limited to: dimethylsulfoxide (DMSO), decylmethylsulfoxide, N,N-dimethyl acetamide, N,N-dimethyl formamide, 2-pyrrolidone, 1-methyl-2-pyrrolidone, 1,5-methyl-2-pyrrolidone, 1-ethyl-2-pyrrolidone, 2-pyrrolidone-5-carboxylic acid, propylene glycol, ethanol, isopropanol, oleic acid, laurocapram (azone), limonene, cieole, diethyl-m-toluamide (DEET), sodium dodecylsulfate, trimethyl phosphine oxide, tetrahydrofurfuryl alcohol, glycerol monolaurate, methyl oleate, propylene glycol monolaurate.

The free base of the racemic form of bupivacaine or ropivacaine or an optically active isomer thereof can be used; a polymorph, solvate or hydrate thereof may also be used. The foams will preferably contain from 1 percent to 30 percent of bupivacaine and/or ropivacaine

### EXAMPLE 17

### Patches and Adhesive Films

Transdermal patch delivery systems were pioneered by Alza Corporation and more than 200 US patents were assigned to Alza for various inventions in connection with patch technologies (ALZA, 2001). Such ALZA patents include, but are not limited to U.S. Pat. No. 5,141,750; U.S. Pat. No. 5,162,042; U.S. Pat. No. 5,284,660; U.S. Pat. No. 5,304,379; U.S. Pat. No. 5,411,740; U.S. Pat. No. 5,508,038; U.S. Pat. No. 5,629,019; U.S. Pat. No. 5,840,327; U.S. Pat. No. 5,879,322; and U.S. Pat. No. 6,348,210. Adhesive matrix type transdermal patch technology was described in U.S. Pat. No. 5,958,447 and U.S. Pat. No. 5,965,154, which patents are hereby incorporated in their entirety by reference. In another embodiment the use of such adhesive matrix type transdermal patches for dermal delivery of bupivacaine is included in the present invention.

Transdermal technologies have been described by Lee C C, 2006, which publication is hereby included in its entirety by reference. Bio-adhesive film for the transdermal delivery of the active ingredients may be developed as described by Padula C et al. 2003, and Padula C et al. 2007, which publications are hereby included in their entirety by reference.

### Method of Manufacture: Patches

The patches of the current invention will use suitable patch manufacturing technology, as described in various Alza patents (see above), as well known to those skilled in the art and as commercially available. For the active ingredients, it will be favorable to include a penetration enhancer into the patch formulation. The drug reservoir in membrane-controlled systems may be filled with a solution or gel composition of the present invention, such solution and gel formulations being described above. If a matrix-type patch system is preferred, said compositions can be used in the polymeric drug reservoir. The free base or a pharmaceutically acceptable salt of the racemic form of bupivacaine or ropivacaine or an optically active isomer thereof can be used; a polymorph, solvate or hydrate thereof may also be used. The solutions or gels in the patches will preferably contain from 1 percent to 20 percent of an active ingredient(s).

### EQUIVALENTS

Based on the present invention, those skilled in the art will recognize, or be able to ascertain, using no more than routine experimentation, that the duration of local anesthesia by bupivacaine or ropivacaine can be extended by the addition of a vasoconstrictor, such as for example epinephrine in concentrations of between 1:200,000 and 1:50,000.

Applied topically, bupivacaine or ropivacaine will also have therapeutic activity for patients suffering from Complex Regional Pain Syndrome (CRPS).

Dermal compositions of the compounds of the present invention, when applied topically to skin adjacent to certain cranial nerves, such as the trigeminal nerve or branches thereof, may be active as medication for patients suffering from certain facial neuropathies, such as for example tic douloureux, and tinnitus that is related to the function of said cranial nerves.

It is known to those skilled in the art that the absorption from dermal compositions is most often heat-sensitive and various heat-generating media can be used with the compositions described here. The use of any heat-generating devices in combination with a composition of the present invention is part of the present invention.

Since the active ingredients can be ionized, dermal iontophoresis delivery systems can be used. These devices use an electric current to “drive” ionized compounds into the skin.

Thermolabile versions of any composition described herein and that generate heat have advantages over other formulations since penetration over stratum corneum is facilitated by increased temperatures. Thermolabile formulations are included in the present invention.

What is claimed is:

1. A dermal composition for external use, comprising a compound selected from the group consisting of 1-butyl-N-(2,6-dimethylphenyl)piperidine-2-carboxamide and 1-propyl-N-(2,6-dimethylphenyl)piperidine-2-carboxamide, or an optically active isomer thereof, or a salt or a solvate or a polymorph thereof, in the form of a topical formulation selected from the group consisting of gels, creams, ointments, aerosols, foams, lotions, pastes, mousse and emulsions.

2. The dermal composition according to claim 1, where said compound is 1-butyl-N-(2,6-dimethylphenyl)piperidine-2-carboxamide (bupivacaine), or an optically active isomer thereof, or a salt or a solvate or a polymorph thereof.

3. The dermal composition according to claim 1, where said compound is 1-propyl-N-(2,6-dimethylphenyl)piperidine-2-carboxamide (ropivacaine), or an optically active isomer thereof, or a salt or a solvate or a polymorph thereof.

4. The dermal composition of claim 1, wherein said compound is present in said formulation in a therapeutically sufficient dose for the treatment of patients suffering from neuropathic pain.

5. The dermal composition of claim 1, wherein said compound is present in said formulation in a therapeutically sufficient dose for the treatment of patients suffering from pruritus.

6. The dermal composition of claim 1, wherein said compound is present in said formulation in a therapeutically sufficient dose for the treatment of patients suffering from nociceptive pain.

7. The dermal composition according to claim 1, wherein said topical formulation is a solution comprising from about 1 percent to about 20 percent of said compound, together with from 5 percent to 85 percent of ethanol, from 0 percent to 75 percent of propylene glycol, from 0 percent to 20 percent of hexylene glycol, from 0 percent to 20 percent of diisopropyl adipate, from 0 percent to 0.3 percent of chelating agents, from 0 percent to 0.3 percent of antioxidants, from 0 percent to 0.5 percent of preservatives, from 0 percent to 50 percent of penetration enhancers and from 0 percent to 2 percent of capsaicin.

8. The dermal composition according to claim 1, wherein said topical formulation is a solution comprising from 1 percent to 20 percent of said compound, from 55 percent to 75 percent of ethanol, 12 percent hexylene glycol, 10 percent diisopropyl adipate, from 0 percent to 0.3 percent of an antioxidant, from 0 percent to 0.5 percent of a preservative and from 0 percent to 2 percent of capsaicin.

9. The dermal composition according to claim 1, wherein said topical formulation is a gel comprising from 1 percent to 20 percent of said compound, from 5 percent to 90 percent of volatile solvents, from 0 percent to 75 percent of non-volatile solvents, from 0.5 percent to 4 percent of polymers, from 0 percent to 0.3 percent of antioxidants, from 0 percent to 0.5 percent of preservatives, from 0 percent to 20 percent of penetration enhancers, from 0 percent to 2 percent of capsaicin and from 0 to 10 percent of a buffer and water to q.s.

10. The dermal composition according to claim 1, wherein said topical formulation is a gel comprising from 1 percent to 20 percent of racemic bupivacaine, from 76 percent to 81 percent of ethanol, 12 percent of hexylene glycol, 2 percent of hydroxyethyl cellulose, from 0 percent to 0.3 percent of an antioxidant, from 0 percent to 0.5 percent of a preservative, from 0 percent to 20 percent of penetration enhancers, from 0 percent to 2 percent of capsaicin and from 0 to 10 percent of a buffer and water to q.s.

11. The dermal composition according to claim 1, wherein said topical formulation is an emollient cream, comprising from 1 percent to 20 percent of said compound, from 0 percent to 50 percent of capric/caprylic triglycerides, from 0 percent to 50 percent of isopropyl myristate, from 0 percent to 25 percent of propylene glycol, from 1 percent to 5 percent of surfactants, from 0.5 percent to 3 percent of polymers, from 0 percent to 0.3 percent of antioxidants, from 0 percent to 0.5 percent of preservatives, from 0 percent to 30 percent of penetration enhancers, from 0 percent to 2 percent of capsaicin, from 0 to 10 percent of a buffer and water to q.s.

12. The dermal composition according to claim 1, wherein said topical formulation is an ointment, comprising from 1 percent to 20 percent of said compound, from 2.5 percent to 10 percent of propylene glycol, from 5 percent to 30 percent of microcrystalline wax, from 0 percent to 20 percent of white wax, from 0.5 percent to 5 percent of surfactants, from 0 percent to 0.1 percent antioxidants, from 0 percent to 20 percent of penetration enhancers, from 0 percent to 2 percent of capsaicin, and mineral oil q.s.

13. The dermal composition according to claim 1, wherein said topical formulation is an aerosol spray, comprising from 1 percent to 20 percent of said compound, from 0 percent to 40 percent of propylene glycol, from 0 percent to 5 percent of surfactants, from 0 percent to 3 percent of polymers, from 0 percent to 0.3 percent of antioxidants, from 0 percent to 0.5 percent of preservatives, from 0 percent to 40 percent of propellants, from 0 percent to 50 percent of penetration enhancers, from 0 percent to 2 percent of capsaicin, and water or buffered water q.s.

14. The dermal composition according to claim 1, wherein said topical formulation is a thermolabile foam, comprising from 1 percent to 20 percent of said compound, from 0 percent to 70 percent ethanol, from 0 percent to 10 percent cetyl alcohol, from 0 percent to 5 percent citric acid, from 0 percent to 10 percent potassium citrate, from 0 percent to 7 percent polysorbates, from 0 percent to 25 percent propylene glycol, from 0 percent to 7 percent stearyl alcohol, from 0 percent to 2 percent of capsaicin and from 0 percent to 3 percent of a corticosteroid

15. The dermal composition according to claim 1, wherein said topical formulation is a thermolabile foam, comprising from 1 percent to 20 percent of bupivacaine, from 0 percent to 70 percent ethanol, from 0 percent to 10 percent cetyl alcohol, from 0 percent to 5 percent citric acid, from 0 percent to 10 percent potassium citrate, from 0 percent to 7 percent polysorbates, from 0 percent to 25 percent propylene glycol, from 0 percent to 7 percent stearyl alcohol, from 0 percent to 2 percent of capsaicin and from 0 percent to 3 percent of a corticosteroid.

16. A dermal composition according to claim 1, being a dermal patch composition, comprising bupivacaine in a concentration from 1 percent to 20 percent and intended for local delivery of bupivacaine into the skin and the tissues below the skin.

17. A dermal composition according to claim 1, being a dermal patch composition, comprising from 1 percent to 20 percent of said N-substituted 2,6-dimethylphenyl-piperidine-carboxamide, from 5 percent to 85 percent of ethanol, from 0 percent to 75 percent propylene glycol, from 0 percent to 20 percent hexylene glycol, from 0 percent to 20 percent diisopropyl adipate, from 0 percent to 0.3 percent of chelating agents, from 0 percent to 0.3 percent of antioxidants, from 0 percent to 0.5 percent of preservatives, from 0 percent to 50 percent of penetration enhancers and from 0 percent to 2 percent of capsaicin.

18. A dermal composition according to claim 1, being a dermal patch composition, comprising from 1 percent to 20 percent of bupivacaine, from 5 percent to 85 percent of ethanol, from 0 percent to 75 percent of propylene glycol, from 0 percent to 20 percent of hexylene glycol, from 0 percent to 20 percent of diisopropyl adipate, from 0 percent to 0.3 percent of chelating agents, from 0 percent to 0.3 percent of antioxidants, from 0 percent to 0.5 percent of preservatives, from 0 percent to 50 percent of penetration enhancers and from 0 percent to 2 percent of capsaicin.

19. The dermal composition according to claim 1, wherein said topical formulation is an emulsion, comprising from 1 percent to 20 percent of said compound, 5 to 25% mineral oil, 2-8% cetostearyl alcohol, 1-5% ceteth-20, 0-2% dimethicone, 0 to 1% buffer, 0 to 0.5% preservatives, 0 to 0.3% antioxidants and from 0 percent to 2 percent of capsaicin.

20. A method of treating a patient suffering from neuropathic pain, comprising topically administering a therapeutically effective amount of a composition comprising a compound selected from the group consisting of 1-butyl-N-(2,6-dimethylphenyl)piperidine-2-carboxamide and 1-propyl-N-(2,6-dimethylphenyl)piperidine-2-carboxamide, or an optically active isomer thereof, or a salt or a solvate or a polymorph thereof.

21. A method of treating a patient suffering from nociceptive pain, comprising topically administering to said patient a therapeutically effective amount of a composition comprising a compound selected from the group consisting of 1-butyl-N-(2,6-dimethylphenyl)piperidine-2-carboxamide and 1-propyl-N-(2,6-dimethylphenyl)piperidine-2-carboxamide, or an optically active isomer thereof, or a salt or a solvate or a polymorph thereof.

22. A method of treating a patient suffering from pruritus, comprising topically administering to said patient a therapeutically effective amount of a composition comprising a compound selected from the group consisting of 1-butyl-N-(2,6-dimethylphenyl)piperidine-2-carboxamide and 1-propyl-N-(2,6-dimethylphenyl)piperidine-2-carboxamide, or an optically active isomer thereof, or a salt or a solvate or a polymorph thereof.
